SEQUENCE LISTING (3 sequences)

SEQ ID NO: 1 - protein, 491 aa, Synthetic construct
MGQIVTFFQEVPHVIEEVMNIVLIALSVLAVLKGLYNFATCGLVGLVTFLLLCGRSCTTSLYKGVYELQTLELNMETLNMTMPLSCTKNNSHHYIMVGNETGLELTLTNTSIINHKFCNLSDAHKKNLYDHALMSIISTFHLSIPNFNQYEAMSCDFNGGKISVQYNLSHSYAGDAANHCGTVANGVLQTFMRMAWGGSYIALDSGRGNWDCIMTSYQYLIIQNTTWEDHCQFSRPSPIGYLGLLSQRTRDIYISRRLLGTFTWTLSDSEGKDTPGGYCLTRWMLIEAELKCFGNTAVAKCNEKHDEEFCDMLRLFDFNKQAIQRLKAEAQMSIQLINKAVNALINDQLIMKNHLRDIMGIPYCNYSKYWYLNHTTTGRTSLPKCWLVSNGSYLNETHFSDDIEQQADNMITEMLQKEYMERQGKTPLGLVDLFVFSTSFYLISIFLHLVKIPTHRHIVGKSCPKPHRLNHMGICSCGLYKQPGVPVKWKR

SEQ ID NO: 2 - DNA, 1473 bp, synthetic construct
  source 1..1473: mol_type = other DNA
atgggccagatcgtgaccttttttcaggaagtgccccacgtcatcgaggaagtgatgaacatcgtcctgatcgccctgagcgtgctggccgtgctgaagggcctgtacaattttgccacatgcggcctggtcggactggtcacatttctgctgctgtgcggcagaagctgcacaacaagcctgtacaagggcgtgtacgagctgcagacactggaactgaacatggaaaccctgaacatgacaatgccactgagctgcaccaagaataacagccaccactacatcatggtcggcaatgagacaggcctggaactgacactgaccaacaccagcatcatcaaccacaagttctgcaatctgagcgacgcccacaagaagaatctgtacgaccacgccctgatgagcatcatcagcacctttcacctgagcatccccaactttaatcagtacgaggccatgagctgcgactttaatggcggcaagatcagcgtgcagtacaatctgagccacagctacgccggcgacgccgccaatcactgcggcacagtggccaatggcgtgctgcagacctttatgagaatggcctggggcggcagctatatcgccctggatagcggcagaggcaattgggattgcatcatgaccagctaccagtacctgatcatccagaatacaacctgggaggaccactgccagtttagcagaccaagcccaatcggctacctgggcctgctgtcccagagaacaagggacatctacatcagcagaaggctgctgggcacctttacatggacactgagcgatagcgagggcaaggatacaccaggcggctactgcctgacaagatggatgctgatcgaggccgagctgaagtgctttggcaatacagccgtggccaagtgcaatgagaagcacgacgaggaattctgcgatatgctgaggctgttcgactttaacaagcaggccatccagagactgaaggccgaggcccagatgtccatccagctgatcaataaggccgtgaacgccctgatcaatgaccagctgatcatgaagaaccacctgagagacatcatgggcatcccatactgcaactacagcaagtactggtatctgaaccacacaacaacaggcagaacaagcctgccaaagtgctggctggtgtccaatggcagctacctgaacgagacacactttagcgacgatatcgagcagcaggccgacaatatgatcacagagatgctgcagaaagaatacatggaaaggcagggcaagacaccactgggcctggtggatctgtttgtgttcagcaccagcttctacctgatcagcatctttctgcacctggtcaagatcccaacacacagacacatcgtgggcaagagctgcccaaagccacacagactgaaccacatgggcatctgcagctgcggcctgtataagcagccaggcgtgccagtgaagtggaagaga

SEQ ID NO: 3 - DNA, 4436 bp, Synthetic construct
  source 1..4436: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccgccaccatgggccagatcgtgaccttttttcaggaagtgccccacgtcatcgaggaagtgatgaacatcgtcctgatcgccctgagcgtgctggccgtgctgaagggcctgtacaattttgccacatgcggcctggtcggactggtcacatttctgctgctgtgcggcagaagctgcacaacaagcctgtacaagggcgtgtacgagctgcagacactggaactgaacatggaaaccctgaacatgacaatgccactgagctgcaccaagaataacagccaccactacatcatggtcggcaatgagacaggcctggaactgacactgaccaacaccagcatcatcaaccacaagttctgcaatctgagcgacgcccacaagaagaatctgtacgaccacgccctgatgagcatcatcagcacctttcacctgagcatccccaactttaatcagtacgaggccatgagctgcgactttaatggcggcaagatcagcgtgcagtacaatctgagccacagctacgccggcgacgccgccaatcactgcggcacagtggccaatggcgtgctgcagacctttatgagaatggcctggggcggcagctatatcgccctggatagcggcagaggcaattgggattgcatcatgaccagctaccagtacctgatcatccagaatacaacctgggaggaccactgccagtttagcagaccaagcccaatcggctacctgggcctgctgtcccagagaacaagggacatctacatcagcagaaggctgctgggcacctttacatggacactgagcgatagcgagggcaaggatacaccaggcggctactgcctgacaagatggatgctgatcgaggccgagctgaagtgctttggcaatacagccgtggccaagtgcaatgagaagcacgacgaggaattctgcgatatgctgaggctgttcgactttaacaagcaggccatccagagactgaaggccgaggcccagatgtccatccagctgatcaataaggccgtgaacgccctgatcaatgaccagctgatcatgaagaaccacctgagagacatcatgggcatcccatactgcaactacagcaagtactggtatctgaaccacacaacaacaggcagaacaagcctgccaaagtgctggctggtgtccaatggcagctacctgaacgagacacactttagcgacgatatcgagcagcaggccgacaatatgatcacagagatgctgcagaaagaatacatggaaaggcagggcaagacaccactgggcctggtggatctgtttgtgttcagcaccagcttctacctgatcagcatctttctgcacctggtcaagatcccaacacacagacacatcgtgggcaagagctgcccaaagccacacagactgaaccacatgggcatctgcagctgcggcctgtataagcagccaggcgtgccagtgaagtggaagagatgatgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

(19) United States
(12) Patent Application Publication
(10) Pub. No.: US 2024/0226265 A9
(43) Pub. Date: Jul. 11, 2024

(54) LASSA VIRUS VACCINE AND USES THEREOF

(71) Applicant: Inovio Pharmaceuticals, Inc., Plymouth Meeting, PA (US)

(72) Inventors: Kate Broderick, San Diego, CA (US); Kathleen A. Cashman, Purcellville, VA (US); Jean D. Boyer, Haddonfield, NJ (US); Scott M. White, Plymouth Meeting, PA (US)

(21) Appl. No.: 18/493,133

(22) Filed: Oct. 24, 2023

Related U.S. Application Data
(60) Provisional application No. 63/380,701, filed on Oct. 24, 2022.

(51) Int. Cl. A61K 39/12; A61K 9/00; A61P 31/14; C07K 14/08

(52) U.S. Cl. CPC A61K 39/12; A61K 9/0019; A61P 31/14; C07K 14/08; A61K 2039/545

(57) ABSTRACT

Described are methods of inducing a protective immune response against Lassa virus comprising administering a prophylactically effective amount of a nucleic acid molecule encoding a Lassa virus glycoprotein precursor (LASV GPC) to a subject in need thereof.

### CROSS-REFERENCE TO RELATED APPLICATIONS

This application claims the benefit of U.S. Provisional Application No. 63/380,701, filed on Oct. 24, 2023, which application is incorporated herein by reference in its entirety.

### SEQUENCE LISTING

The instant application contains a Sequence Listing which has been submitted electronically in .XML format and is hereby incorporated by reference in its entirety. Said .XML copy, created on Oct. 24, 2023 is named “104409000898_Sequence Listing.xml” and is 10,044 bytes in size. The sequence listing contained in this .XML file is part of the specification and is hereby incorporated by reference herein in its entirety.

### TECHNICAL FIELD

Disclosed herein are methods of inducing a protective immune response against Lassa virus and methods of preventing Lassa virus infection. The methods involve administering a prophylactically effective amount of a nucleic acid molecule encoding a Lassa virus glycoprotein precursor (LASV GPC) to a subject in need thereof.

### BACKGROUND

Lassa fever (LF), also known as Lassa Hemorrhagic Fever, is a type of viral hemorrhagic fever caused by the Lassa virus (LASV). Between 100,000-300,000 people are infected with Lassa virus each year, with approximately 5,000 hospitalized patient deaths. The overall mortality rate is estimated to be 1%, but during outbreaks, mortality can climb as high as 50%. The mortality rate is greater than 80% in pregnant women during their third trimester; fetal death also occurs in nearly all cases. The main feature of fatal illness is impaired or delayed cellular immunity leading to fulminant viraemia.

The majority of infections with LASV are asymptomatic to mild and never progress beyond a “flu-like” illness. Symptoms generally include fever, weakness, sore throat, headache, vomiting and muscle pain. In some, LF progresses to the severe hemorrhagic form of the disease, which is associated with significant morbidity and mortality. Symptoms of this severe illness includes hemorrhage, respiratory distress and fluid in the pulmonary cavity. Within two weeks of the onset of symptoms multi-organ complications and/or organ failure can occur resulting in death. Hearing loss (ranging from mild to severe) and encephalopathy have also been documented in LF survivors.

There is a need for methods of inducing protective immune response against LASV infection.

### SUMMARY

Provided herein are methods of inducing a protective immune response against Lassa virus in a subject in need thereof. The methods involve administering a prophylactically effective amount of a nucleic acid molecule encoding the amino acid sequence of SEQ ID NO: 1 to the subject. Also provided are uses of a prophylactically effective amount of a nucleic acid molecule encoding the amino acid sequence of SEQ ID NO: 1 in a method of inducing a protective immune response against Lassa virus in a subject in need thereof.

In certain embodiments, the nucleic acid molecule comprises SEQ ID NO: 2. The nucleic acid molecule may be an expression vector. In some embodiments, the expression vector is a plasmid, such as, for example, pGX9808. According to some embodiments, the nucleic acid molecule encoding the amino acid sequence of SEQ ID NO: 1 is operably linked to a promoter.

In certain embodiments, the nucleic acid molecule is formulated for administration in a buffer. The buffer may comprise sodium chloride and sodium citrate. The buffer may, for example, comprise 150 mM sodium chloride and 15 mM sodium citrate at pH7. In certain embodiments, INO-4500 is administered to the subject.

In certain embodiments, administration of the nucleic acid molecule is by injection, optionally intradermal injection. In some embodiments, administration of the nucleic acid molecule also involves electroporation.

In certain embodiments, the subject is administered a dose of 2 mg of the expression vector. In further embodiments, the subject is administered an initial dose of 2 mg of the expression vector and a second dose of 2 mg of the expression vector. The second dose may be administered to the subject 23 to 33 days after the initial dose.

In certain embodiments, the method provides a statistically significant increase in overall cellular response rate in the subject relative to baseline as measured by a Lassa virus glycoprotein precursor (LASV GPC) interferon-γ (IFN-γ) ELISpot assay six weeks and/or twelve weeks after the initial dose.

In certain embodiments, the method provides a statistically significant increase in overall cellular response rate in a population of subjects administered the nucleic acid molecule as measured by a Lassa virus glycoprotein precursor (LASV GPC) interferon-γ (IFN-γ) ELISpot assay relative to a population of subjects not administered the nucleic acid molecule. According to some embodiments, the increase is about 40% to about 70%.

In certain embodiments, the method induces: (i) a cellular immune response in about 70% of a population of subjects as measured by a Lassa virus glycoprotein precursor (LASV GPC) interferon-γ (IFN-γ) ELISpot assay six weeks following administration of the initial dose; (ii) a cellular immune response in about 83% of a population of subjects as measured by a Lassa virus glycoprotein precursor (LASV GPC) interferon-γ (IFN-γ) ELISpot assay twelve weeks following administration of the initial dose; (iii) a cellular immune response in about 80% of a population of subjects as measured by a multiplex-based cytokine/chemokine assay for interleukin-2 (IL2), interferon-γ (IFN-γ), tumor necrosis factor-α (TNF-α), IFN-γ-induced protein-10 (IP10), and monokine-induced-by-IFN-γ (MIG) six weeks following administration of the initial dose; and/or (iv) a cellular immune response in about 80% of a population of subjects as measured by a multiplex-based cytokine/chemokine assay for interleukin-2 (IL2), interferon-γ (IFN-γ), tumor necrosis factor-α (TNF-α), IFN-γ-induced protein-10 (IP10), and monokine-induced-by-IFN-γ (MIG) twelve weeks following administration of the initial dose.

In certain embodiments, the method provides no sensoneuronal hearing loss in the subject.

### BRIEF DESCRIPTION OF THE DRAWINGS

FIG. 1 provides a plasmid map of pGX9808. DNA plasmid pGX9808 is a DNA plasmid expressing the Lassa virus glycoprotein precursor (LASV GPC), driven by a human CMV promoter (hCMV promoter) with the bovine growth hormone 3′end poly-adenylation signal (bGH polyA). The pGX0001 backbone includes the kanamycin resistance gene (KanR) and plasmid origin of replication (pUC ori).

FIGS. 2A-2D show dose-ranging immunogenicity & protective efficacy of INO-4500 in non-human primates (NHPs). NHPs were immunized at Weeks 0 and 4 with 2 mg, 1 mg, or 0.1 mg INO-4500, and then challenged with lethal LASV at WK 8. FIG. 2A illustrates survival data showing percent survival of 100% (6/6), 66% (4/6), 50% (3/6) or 0% (0/6) for the 2 mg (circle), 1 mg (square), 0.1 mg (triangle), or control (inverted triangle) groups, respectively. PBMC and sera samples were collected at pre-challenge (WK 0), and 2-weeks post immunizations (WKs 2 and 6). FIG. 2B provides cellular responses to LASV GPC measured by IFNγ ELISpot assay. FIG. 2C shows binding IgG antibody responses to LASV GPC measured by endpoint titer ELISA. FIG. 2D shows neutralizing activity measured by pseudovirus assay at 1:20 sera dilution. Bars and whiskers represent median min to max for each group and symbols represent individual animals. ***p<0.0001 indicate significant difference vs week 0 and $p<0.05 indicate significant difference vs control by 2-way ANOVA with Dunnett's multiple comparison test (ELISpot and ELISA) or by ANOVA with Kruskal-Wallis test (neutralization).

FIGS. 3A-3C detail 1-year durability of immunogenicity and protective efficacy of INO-4500 in NHPs. NHPs were immunized at weeks 0 and 4 with 2 mg or 1 mg INO-4500, then challenged with lethal LASV at 12 months. FIG. 3C shows survival data showing percent survival of 100% (6/6) for the 2 mg (circle) and 1 mg (square) INO-4500 dose groups, and 0% survival for the control group (inverted triangle). PBMC and sera samples were collected at week 0 pre-immunization (WK0), two weeks after first (WK2) or second (WK6) immunization, and the indicated months after start of immunization (M4, M6, M9, M12). FIG. 3A details cellular responses to LASV GPC measured by IFNγ ELISpot assay. FIG. 3B shows humoral responses measured as IgG binding endpoint titers against LASV GPC by ELISA. Bars and whiskers represent median min to max for each group and symbols represent individual animals. ****p<000.1, ***p<0.001, **p<0.001, indicated significant differences vs. baseline (WK0) by 2-way ANOVA with Dunnett's multiple comparison test. Survival data showing percent survival of 100% (6/6) for the 2 mg (circle) and 1 mg (square) INO-4500 dose groups, and 0% survival for the control group (inverted triangle).

FIGS. 4A-4D show immunogenicity and protective efficacy of 1 or 2 INO-4500 immunizations in NHPs. NHPs were immunized at WK 0 (one dose) or WKs 0 and 4 (two doses) with 2 mg of INO-4500 followed by EP and challenged with lethal LASV at WK 8. FIG. 4A provides survival data showing percent survival of 100% for NHPs after two (circle, n of 6) or one immunization (square, n of 6) with INO-4500, and 0% survival for the control group (inverted triangle, n of 6). PBMC and sera samples were collected at the indicated time-points. FIG. 4B shows cellular responses to clade IV LASV GPC peptides measured by IFNγ ELISpot assay. FIG. 4C shows humoral responses measured as IgG binding endpoint titers against LASV GPC by ELISA. FIG. 4D shows serum LASV neutralizing antibodies by PRNT assay. Results are expressed as the reciprocal of the serum dilution resulting in 50% reduction in plaques compared to virus-only control wells. Bars and whiskers (FIGS. 4B and 4C) represent median min to max for each group and symbols represent individual animals. Lines (FIG. 4D) represent the mean values within each group (circle: two-dose; square: one-dose; triangle: control group), and individual symbols represent the mean values of individual NHPs.

FIGS. 5A-5E show cellular and humoral immune responses against heterologous LASV clades for INO-4500 immunized NHPs. NHPs previously immunized with INO-4500 at weeks (WKs) 0, 4, and 8 received a booster immunization at WK 37. In FIG. 5A, PBMC samples were collected at pre-boost (WK 37) and post-boost (WK 39) and cellular responses to peptides spanning full-length GPCs of the indicated LASV clades were measured by IFNγ ELISpot. Sera samples collected at the indicated timepoints were evaluated for IgG binding endpoint titers (FIGS. 5B, D) or pseudovirus neutralizing activity (FIGS. 5C, 5E) against the indicated clades of LASV. Bars and whiskers represent median min to max each group and symbols represent individual animals. * p<0.05, ****p<0.001 indicate significant difference vs WK 0 by 2-way ANOVA; $p<0.05 indicate significant difference between WK 37 and WK 39 by Mann-Whitney U-test.

FIG. 6 illustrates IFNγ ELISpot responses against LASV peptides by study group. PBMC from volunteers collected at different time points during the trial were stimulated with two different pools of LASV peptides, Pool 1 and Pool 2. Cellular responses were measured by IFNγ ELISpot. Box and whiskers represent median min to max IFNγ SFUs for each group and symbols represent individual subjects. Multiple comparisons were performed by Kruskal-Wallis with Dunn's post-test. Unpaired comparisons were performed by Mann Whitney test (*<0.05, **<0.005, ***<0.0005, ****<0.0001).

### DETAILED DESCRIPTION OF ILLUSTRATIVE EMBODIMENTS

The disclosed methods may be understood more readily by reference to the following detailed description, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

When a range of numerical values is expressed, another embodiment includes from the one particular value and/or to the other particular value. Further, reference to values stated in ranges include each and every value within that range. All ranges are inclusive and combinable. When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed methods, which are, for clarity, described herein in the context of separate embodiments, may also be provided in combination in a single embodiment. Conversely, various features of the disclosed methods that are, for brevity, described in the context of a single embodiment, may also be provided separately or in any subcombination.

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, the singular forms “a,” “an,” and “the” include the plural.

### Certain Terminology

As used herein, the term “about” when used in reference to numerical ranges, cutoffs, or specific values is used to indicate that the recited values may vary by up to as much as 10% from the listed value. Thus, the term “about” is used to encompass variations of ±10% or less, variations of ±5% or less, variations of ±1% or less, variations of ±0.5% or less, or variations of ±0.1% or less from the specified value.

As used herein, the term “at least one” means “one or more.”

As used herein, the term “subject” as used herein refers to any animal, but in particular humans. Thus, the methods are applicable to human and nonhuman animals, although preferably used most preferably with humans. “Subject” and “patient” are used interchangeably herein.

As used herein, the term “comprising” is intended to include examples encompassed by the terms “consisting essentially of” and “consisting of”; similarly, the term “consisting essentially of” is intended to include examples encompassed by the term “consisting of.”

As used herein, the term “coding sequence” or “encoding nucleic acid” may mean refers to the nucleic acid (RNA or DNA molecule) that comprise a nucleotide sequence which encodes a polypeptide. The coding sequence may further include initiation and termination signals operably linked to regulatory elements including a promoter and polyadenylation signal capable of directing expression in the cells of an individual or mammal to whom the nucleic acid is administered. The coding sequence may further include sequences that encode signal peptides, e.g., an IgE leader sequence.

As used herein, the term “nucleic acid” or “oligonucleotide” or “polynucleotide” may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

As used herein, the term “operably linked” may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function,

As used herein, the term “promoter” may mean a synthetic or naturally derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents.

As used herein, the term “vector” may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

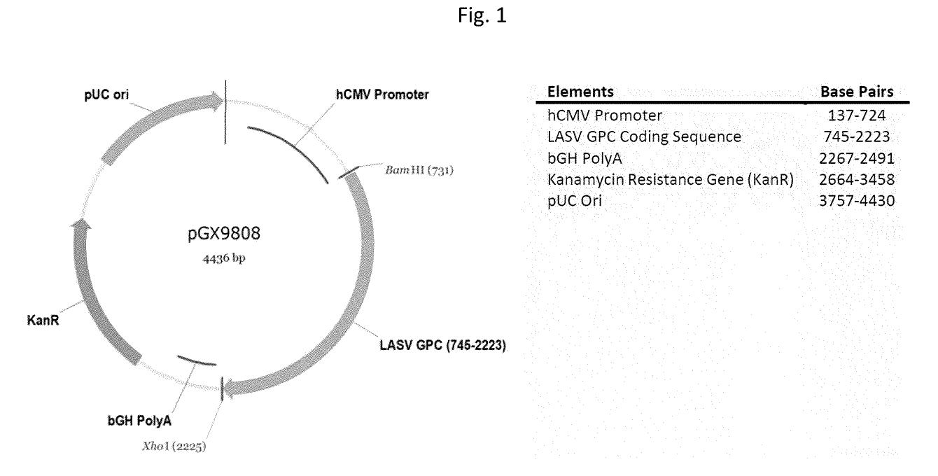

“Investigational product” as used herein refers to a pharmaceutical form of an active ingredient or placebo being tested or used as a reference in the study, whether blinded or unblinded. The investigational product INO-4500 contains pGX9808 in 1×SSC buffer (150 mM sodium chloride and 15 mM sodium citrate, pH 7.0). The active pharmaceutical ingredient, pGX9808, of INO-4500 is a DNA plasmid designed to express the Lassa virus (Josiah strain) glycoprotein precursor (LASV GPC), driven by a human CMV promoter (hCMV promoter) with the bovine growth hormone 3′end poly-adenylation signal (bGH polyA). The plasmid backbone (pGX0001) includes the kanamycin resistance gene (KanR) and plasmid origin of replication (pUC ori) (FIG. 1). The amino acid and nucleotide sequences for LASV GPC are:

| LASV GPC Amino Acid Sequence | |
|---|---|
| (SEQ ID NO: 1) | |
| 1 | MGQIVTFFQE VPHVIEEVMN IVLIALSVLA VLKGLYNFAT CGLVGLVTFL |
| 51 | LLCGRSCTTS LYKGVYELQT LELNMETLNM TMPLSCTKNN SHHYIMVGNE |
| 101 | TGLELTLTNT SIINHKFCNL SDAHKKNLYD HALMSIISTF HLSIPNFNQY |
| 151 | EAMSCDFNGG KISVQYNLSH SYAGDAANHC GTVANGVLQT FMRMAWGGSY |
| 201 | IALDSGRGNW DCIMTSYQYL IIQNTTWEDH CQFSRPSPIG YLGLLSQRTR |
| 251 | DIYISRRLLG TFTWTLSDSE GKDTPGGYCL TRWMLIEAEL KCFGNTAVAK |
| 301 | CNEKHDEEFC DMLRLFDFNK QAIQRLKAEA QMSIQLINKA VNALINDQLI |
| 351 | MKNHLRDIMG IPYCNYSKYW YLNHTTTGRT SLPKCWLVSN GSYLNETHFS |
| 401 | DDIEQQADNM ITEMLQKEYM ERQGKTPLGL VDLFVFSTSF YLISIFLHLV |
| 451 | KIPTHRHIVG KSCPKPHRLN HMGICSCGLY KQPGVPVKWK R** |
| Nucleotide sequence encoding LASV GPC | |
| (SEQ ID NO: 2) | |
| ATGGGCCAGATCGTGACCTTTTTTCAGGAAGTGCCCCACGTCATCGAGGAAGTGATGAAC | |
| ATCGTCCTGATCGCCCTGAGCGTGCTGGCCGTGCTGAAGGGCCTGTACAATTTTGCCACA | |
| TGCGGCCTGGTCGGACTGGTCACATTTCTGCTGCTGTGCGGCAGAAGCTGCACAACAAGC | |
| CTGTACAAGGGCGTGTACGAGCTGCAGACACTGGAACTGAACATGGAAACCCTGAACATG | |
| ACAATGCCACTGAGCTGCACCAAGAATAACAGCCACCACTACATCATGGTCGGCAATGAG | |
| ACAGGCCTGGAACTGACACTGACCAACACCAGCATCATCAACCACAAGTTCTGCAATCTG | |
| AGCGACGCCCACAAGAAGAATCTGTACGACCACGCCCTGATGAGCATCATCAGCACCTTT | |
| CACCTGAGCATCCCCAACTTTAATCAGTACGAGGCCATGAGCTGCGACTTTAATGGCGGC | |
| AAGATCAGCGTGCAGTACAATCTGAGCCACAGCTACGCCGGCGACGCCGCCAATCACTGC | |
| GGCACAGTGGCCAATGGCGTGCTGCAGACCTTTATGAGAATGGCCTGGGGCGGCAGCTAT | |
| ATCGCCCTGGATAGCGGCAGAGGCAATTGGGATTGCATCATGACCAGCTACCAGTACCTG | |
| ATCATCCAGAATACAACCTGGGAGGACCACTGCCAGTTTAGCAGACCAAGCCCAATCGGC | |
| TACCTGGGCCTGCTGTCCCAGAGAACAAGGGACATCTACATCAGCAGAAGGCTGCTGGGC | |
| ACCTTTACATGGACACTGAGCGATAGCGAGGGCAAGGATACACCAGGCGGCTACTGCCTG | |
| ACAAGATGGATGCTGATCGAGGCCGAGCTGAAGTGCTTTGGCAATACAGCCGTGGCCAAG | |
| TGCAATGAGAAGCACGACGAGGAATTCTGCGATATGCTGAGGCTGTTCGACTTTAACAAG | |
| CAGGCCATCCAGAGACTGAAGGCCGAGGCCCAGATGTCCATCCAGCTGATCAATAAGGCC | |
| GTGAACGCCCTGATCAATGACCAGCTGATCATGAAGAACCACCTGAGAGACATCATGGGC | |
| ATCCCATACTGCAACTACAGCAAGTACTGGTATCTGAACCACACAACAACAGGCAGAACA | |
| AGCCTGCCAAAGTGCTGGCTGGTGTCCAATGGCAGCTACCTGAACGAGACACACTTTAGC | |
| GACGATATCGAGCAGCAGGCCGACAATATGATCACAGAGATGCTGCAGAAAGAATACATG | |
| GAAAGGCAGGGCAAGACACCACTGGGCCTGGTGGATCTGTTTGTGTTCAGCACCAGCTTC | |
| TACCTGATCAGCATCTTTCTGCACCTGGTCAAGATCCCAACACACAGACACATCGTGGGC | |
| AAGAGCTGCCCAAAGCCACACAGACTGAACCACATGGGCATCTGCAGCTGCGGCCTGTAT | |
| AAGCAGCCAGGCGTGCCAGTGAAGTGGAAGAGA | |
| pGX9808 Single Strand DNA Sequence | |
| (SEQ ID NO: 3) | |
| 1 | GCTGCTTCGC GATGTACGGG CCAGATATAC GCGTTGACAT TGATTATTGA |
| 51 | CTAGTTATTA ATAGTAATCA ATTACGGGGT CATTAGTTCA TAGCCCATAT |
| 101 | ATGGAGTTCC GCGTTACATA ACTTACGGTA AATGGCCCGC CTGGCTGACC |
| 151 | GCCCAACGAC CCCCGCCCAT TGACGTCAAT AATGACGTAT GTTCCCATAG |
| 201 | TAACGCCAAT AGGGACTTTC CATTGACGTC AATGGGTGGA GTATTTACGG |
| 251 | TAAACTGCCC ACTTGGCAGT ACATCAAGTG TATCATATGC CAAGTACGCC |
| 301 | CCCTATTGAC GTCAATGACG GTAAATGGCC CGCCTGGCAT TATGCCCAGT |
| 351 | ACATGACCTT ATGGGACTTT CCTACTTGGC AGTACATCTA CGTATTAGTC |
| 401 | ATCGCTATTA CCATGGTGAT GCGGTTTTGG CAGTACATCA ATGGGCGTGG |
| 451 | ATAGCGGTTT GACTCACGGG GATTTCCAAG TCTCCACCCC ATTGACGTCA |
| 501 | ATGGGAGTTT GTTTTGGCAC CAAAATCAAC GGGACTTTCC AAAATGTCGT |
| 551 | AACAACTCCG CCCCATTGAC GCAAATGGGC GGTAGGCGTG TACGGTGGGA |
| 601 | GGTCTATATA AGCAGAGCTC TCTGGCTAAC TAGAGAACCC ACTGCTTACT |
| 651 | GGCTTATCGA AATTAATACG ACTCACTATA GGGAGACCCA AGCTGGCTAG |
| 701 | CGTTTAAACT TAAGCTTGGT ACCGAGCTCG GATCCGCCGC CACCATGGGC |
| 751 | CAGATCGTGA CCTTTTTTCA GGAAGTGCCC CACGTCATCG AGGAAGTGAT |
| 801 | GAACATCGTC CTGATCGCCC TGAGCGTGCT GGCCGTGCTG AAGGGCCTGT |
| 851 | ACAATTTTGC CACATGCGGC CTGGTCGGAC TGGTCACATT TCTGCTGCTG |
| 901 | TGCGGCAGAA GCTGCACAAC AAGCCTGTAC AAGGGCGTGT ACGAGCTGCA |
| 951 | GACACTGGAA CTGAACATGG AAACCCTGAA CATGACAATG CCACTGAGCT |
| 1001 | GCACCAAGAA TAACAGCCAC CACTACATCA TGGTCGGCAA TGAGACAGGC |
| 1051 | CTGGAACTGA CACTGACCAA CACCAGCATC ATCAACCACA AGTTCTGCAA |
| 1101 | TCTGAGCGAC GCCCACAAGA AGAATCTGTA CGACCACGCC CTGATGAGCA |
| 1151 | TCATCAGCAC CTTTCACCTG AGCATCCCCA ACTTTAATCA GTACGAGGCC |
| 1201 | ATGAGCTGCG ACTTTAATGG CGGCAAGATC AGCGTGCAGT ACAATCTGAG |
| 1251 | CCACAGCTAC GCCGGCGACG CCGCCAATCA CTGCGGCACA GTGGCCAATG |
| 1301 | GCGTGCTGCA GACCTTTATG AGAATGGCCT GGGGCGGCAG CTATATCGCC |
| 1351 | CTGGATAGCG GCAGAGGCAA TTGGGATTGC ATCATGACCA GCTACCAGTA |
| 1401 | CCTGATCATC CAGAATACAA CCTGGGAGGA CCACTGCCAG TTTAGCAGAC |
| 1451 | CAAGCCCAAT CGGCTACCTG GGCCTGCTGT CCCAGAGAAC AAGGGACATC |
| 1501 | TACATCAGCA GAAGGCTGCT GGGCACCTTT ACATGGACAC TGAGCGATAG |
| 1551 | CGAGGGCAAG GATACACCAG GCGGCTACTG CCTGACAAGA TGGATGCTGA |
| 1601 | TCGAGGCCGA GCTGAAGTGC TTTGGCAATA CAGCCGTGGC CAAGTGCAAT |
| 1651 | GAGAAGCACG ACGAGGAATT CTGCGATATG CTGAGGCTGT TCGACTTTAA |
| 1701 | CAAGCAGGCC ATCCAGAGAC TGAAGGCCGA GGCCCAGATG TCCATCCAGC |
| 1751 | TGATCAATAA GGCCGTGAAC GCCCTGATCA ATGACCAGCT GATCATGAAG |
| 1801 | AACCACCTGA GAGACATCAT GGGCATCCCA TACTGCAACT ACAGCAAGTA |
| 1851 | CTGGTATCTG AACCACACAA CAACAGGCAG AACAAGCCTG CCAAAGTGCT |
| 1901 | GGCTGGTGTC CAATGGCAGC TACCTGAACG AGACACACTT TAGCGACGAT |
| 1951 | ATCGAGCAGC AGGCCGACAA TATGATCACA GAGATGCTGC AGAAAGAATA |
| 2001 | CATGGAAAGG CAGGGCAAGA CACCACTGGG CCTGGTGGAT CTGTTTGTGT |
| 2051 | TCAGCACCAG CTTCTACCTG ATCAGCATCT TTCTGCACCT GGTCAAGATC |
| 2101 | CCAACACACA GACACATCGT GGGCAAGAGC TGCCCAAAGC CACACAGACT |
| 2151 | GAACCACATG GGCATCTGCA GCTGCGGCCT GTATAAGCAG CCAGGCGTGC |
| 2201 | CAGTGAAGTG GAAGAGATGA TGACTCGAGT CTAGAGGGCC CGTTTAAACC |
| 2251 | CGCTGATCAG CCTCGACTGT GCCTTCTAGT TGCCAGCCAT CTGTTGTTTG |
| 2301 | CCCCTCCCCC GTGCCTTCCT TGACCCTGGA AGGTGCCACT CCCACTGTCC |
| 2351 | TTTCCTAATA AAATGAGGAA ATTGCATCGC ATTGTCTGAG TAGGTGTCAT |
| 2401 | TCTATTCTGG GGGGTGGGGT GGGGCAGGAC AGCAAGGGGG AGGATTGGGA |
| 2451 | AGACAATAGC AGGCATGCTG GGGATGCGGT GGGCTCTATG GCTTCTACTG |
| 2501 | GGCGGTTTTA TGGACAGCAA GCGAACCGGA ATTGCCAGCT GGGGCGCCCT |
| 2551 | CTGGTAAGGT TGGGAAGCCC TGCAAAGTAA ACTGGATGGC TTTCTTGCCG |
| 2601 | CCAAGGATCT GATGGCGCAG GGGATCAAGC TCTGATCAAG AGACAGGATG |
| 2651 | AGGATCGTTT CGCATGATTG AACAAGATGG ATTGCACGCA GGTTCTCCGG |
| 2701 | CCGCTTGGGT GGAGAGGCTA TTCGGCTATG ACTGGGCACA ACAGACAATC |
| 2751 | GGCTGCTCTG ATGCCGCCGT GTTCCGGCTG TCAGCGCAGG GGCGCCCGGT |
| 2801 | TCTTTTTGTC AAGACCGACC TGTCCGGTGC CCTGAATGAA CTGCAAGACG |
| 2851 | AGGCAGCGCG GCTATCGTGG CTGGCCACGA CGGGCGTTCC TTGCGCAGCT |
| 2901 | GTGCTCGACG TTGTCACTGA AGCGGGAAGG GACTGGCTGC TATTGGGCGA |
| 2951 | AGTGCCGGGG CAGGATCTCC TGTCATCTCA CCTTGCTCCT GCCGAGAAAG |
| 3001 | TATCCATCAT GGCTGATGCA ATGCGGCGGC TGCATACGCT TGATCCGGCT |
| 3051 | ACCTGCCCAT TCGACCACCA AGCGAAACAT CGCATCGAGC GAGCACGTAC |
| 3101 | TCGGATGGAA GCCGGTCTTG TCGATCAGGA TGATCTGGAC GAAGAGCATC |
| 3151 | AGGGGCTCGC GCCAGCCGAA CTGTTCGCCA GGCTCAAGGC GAGCATGCCC |
| 3201 | GACGGCGAGG ATCTCGTCGT GACCCATGGC GATGCCTGCT TGCCGAATAT |
| 3251 | CATGGTGGAA AATGGCCGCT TTTCTGGATT CATCGACTGT GGCCGGCTGG |
| 3301 | GTGTGGCGGA CCGCTATCAG GACATAGCGT TGGCTACCCG TGATATTGCT |
| 3351 | GAAGAGCTTG GCGGCGAATG GGCTGACCGC TTCCTCGTGC TTTACGGTAT |
| 3401 | CGCCGCTCCC GATTCGCAGC GCATCGCCTT CTATCGCCTT CTTGACGAGT |
| 3451 | TCTTCTGAAT TATTAACGCT TACAATTTCC TGATGCGGTA TTTTCTCCTT |
| 3501 | ACGCATCTGT GCGGTATTTC ACACCGCATC AGGTGGCACT TTTCGGGGAA |
| 3551 | ATGTGCGCGG AACCCCTATT TGTTTATTTT TCTAAATACA TTCAAATATG |
| 3601 | TATCCGCTCA TGAGACAATA ACCCTGATAA ATGCTTCAAT AATAGCACGT |
| 3651 | GCTAAAACTT CATTTTTAAT TTAAAAGGAT CTAGGTGAAG ATCCTTTTTG |
| 3701 | ATAATCTCAT GACCAAAATC CCTTAACGTG AGTTTTCGTT CCACTGAGCG |
| 3751 | TCAGACCCCG TAGAAAAGAT CAAAGGATCT TCTTGAGATC CTTTTTTTCT |
| 3801 | GCGCGTAATC TGCTGCTTGC AAACAAAAAA ACCACCGCTA CCAGCGGTGG |
| 3851 | TTTGTTTGCC GGATCAAGAG CTACCAACTC TTTTTCCGAA GGTAACTGGC |
| 3901 | TTCAGCAGAG CGCAGATACC AAATACTGTT CTTCTAGTGT AGCCGTAGTT |
| 3951 | AGGCCACCAC TTCAAGAACT CTGTAGCACC GCCTACATAC CTCGCTCTGC |
| 4001 | TAATCCTGTT ACCAGTGGCT GCTGCCAGTG GCGATAAGTC GTGTCTTACC |
| 4051 | GGGTTGGACT CAAGACGATA GTTACCGGAT AAGGCGCAGC GGTCGGGCTG |
| 4101 | AACGGGGGGT TCGTGCACAC AGCCCAGCTT GGAGCGAACG ACCTACACCG |
| 4151 | AACTGAGATA CCTACAGCGT GAGCTATGAG AAAGCGCCAC GCTTCCCGAA |
| 4201 | GGGAGAAAGG CGGACAGGTA TCCGGTAAGC GGCAGGGTCG GAACAGGAGA |
| 4251 | GCGCACGAGG GAGCTTCCAG GGGGAAACGC CTGGTATCTT TATAGTCCTG |
| 4301 | TCGGGTTTCG CCACCTCTGA CTTGAGCGTC GATTTTTGTG ATGCTCGTCA |
| 4351 | GGGGGGCGGA GCCTATGGAA AAACGCCAGC AACGCGGCCT TTTTACGGTT |
| 4401 | CCTGGCCTTT TGCTGGCCTT TTGCTCACAT GTTCTT |

Provided herein are methods of inducing a protective immune response against Lassa virus in a subject in need thereof, the methods comprising, consisting of, or consisting essentially of: administering a prophylactically effective amount of a nucleic acid molecule encoding the amino acid sequence of SEQ ID NO: 1 or a nucleic acid molecule encoding an amino acid sequence 95%, 96%, 97%, 98%, or 99% identical to SEQ ID NO: 1 to the subject.

Also provided are uses of a prophylactically effective amount of a nucleic acid molecule encoding the amino acid sequence of SEQ ID NO: 1 or a nucleic acid molecule encoding an amino acid sequence 95%, 96%, 97%, 98%, or 99% identical to SEQ ID NO: 1 in a method of inducing a protective immune response against Lassa virus in a subject in need thereof.

Further provided are uses of a prophylactically effective amount of a nucleic acid molecule encoding the amino acid sequence of SEQ ID NO: 1 or a nucleic acid molecule encoding an amino acid sequence 95%, 96%, 97%, 98%, or 99% identical to SEQ ID NO: 1 in a method of manufacturing a medicament for inducing a protective immune response against Lassa virus in a subject in need thereof.

In certain embodiments, the nucleic acid molecule comprises the nucleotide sequence of SEQ ID NO: 2 or a nucleotide sequence that is 95%, 96%, 97%, 98%, or 99% identical to SEQ ID NO: 2. The nucleic acid molecule may be an expression vector. In some embodiments, the expression vector is a DNA plasmid, such as, for example, pGX9808. According to some embodiments, the nucleic acid molecule encoding the amino acid sequence of SEQ ID NO: 1 or a nucleic acid molecule encoding an amino acid sequence 95%, 96%, 97%, 98%, or 99% identical to SEQ ID NO: 1 is operably linked to a promoter.

The pharmaceutical compositions according to the present invention comprise about 500 nanograms to about 3000 micrograms of DNA. In some embodiments, the pharmaceutical composition is a vaccine. In some embodiments, pharmaceutical compositions according to the present invention comprise about 1000 nanogram to about 2500 micrograms of DNA. In some preferred embodiments, the pharmaceutical compositions contain about 1000 micrograms to about 2000 micrograms of DNA. In some preferred embodiments, the pharmaceutical compositions contain about 0.1 to about 2 milligrams of DNA. In some embodiments, the pharmaceutical compositions contain about 1 mg of DNA. In some embodiments, the pharmaceutical compositions contain about 2 mg of DNA. In certain embodiments, the plasmid is formulated at a concentration of about 1 mg/ml to about 20 mg/ml. In certain embodiments, the plasmid is formulated at a concentration of 10 mg/ml. In certain embodiments, the plasmid is formulated at a concentration of 20 mg/ml.

The nucleic acid molecule may be formulated according to the mode of administration to be used. In cases where the nucleic acid molecule is formulated as an injectable pharmaceutical composition, it is sterile, pyrogen free and particulate free. An isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation. In certain embodiments, the nucleic acid molecule is formulated for administration in a buffer. The buffer may comprise sodium chloride and sodium citrate. The buffer may, for example, comprise 150 mM sodium chloride and 15 mM sodium citrate at pH7. In some preferred embodiments, the pharmaceutical compositions contain about 100 micrograms of DNA. In certain embodiments, the compositions are formulated at a concentration of about 0.1 mg/ml pGX9808 in 1×SSC buffer (150 mM sodium chloride and 15 mM sodium citrate, pH 7.0). In some preferred embodiments, the pharmaceutical compositions contain about 1 milligram of DNA. In certain embodiments, the compositions are formulated at a concentration of 10 mg/ml pGX9808 in 1×SSC buffer (150 mM sodium chloride and 15 mM sodium citrate, pH 7.0). In some preferred embodiments, the pharmaceutical compositions contain about 2 milligrams of DNA. In certain embodiments, the INO-4500 is formulated at a concentration of 20 mg/ml pGX9808 in 1×SSC buffer (150 mM sodium chloride and 15 mM sodium citrate, pH 7.0). In certain embodiments, INO-4500 is administered to the subject.

In certain embodiments, the subject is administered a dose of 2 mg of the expression vector. In further embodiments, the subject is administered an initial dose of 0.1 mg, 1 mg, or 2 mg of the expression vector and a second dose of 0.1 mg, 1 mg, or 2 mg of the expression vector. The second dose may be administered to the subject 23 to 33 days after the initial dose. In further embodiments, INO-4500 is administered to the subject two times over the course of four weeks. In still further embodiments, a first dose of INO-4500 is administered on Day 0, and the second dose of INO-4500 is administered at Week 4±5 days.

The nucleic acid molecule administered in accordance with the methods and uses of the invention may be delivered using any of several well-known technologies including DNA injection (also referred to as DNA vaccination). Routes of administration include, but are not limited to, intramuscular, intradermal, and subcutaneous administration. In some embodiments, the nucleic acid molecule is administered to the subject by intradermal injection. In certain embodiments, the nucleic acid molecule is administered to the subject by intradermal injection followed by electroporation.

Examples of electroporation devices and electroporation methods preferred for facilitating delivery of the DNA vaccines, include those described in U.S. Pat. No. 7,245,963 by Draghia-Akli, et al, U.S. Patent Pub. 2005/0052630 submitted by Smith, et al., the contents of which are hereby incorporated by reference in their entirety. Also preferred, are electroporation devices and electroporation methods for facilitating delivery of the DNA vaccines provided in co-pending and co-owned U.S. patent application Ser. No. 11/874,072, filed Oct. 17, 2007, which claims the benefit under 35 USC 1 19(e) to U.S. Provisional Application Ser. No. 60/852,149, filed Oct. 17, 2006, and 60/978,982, filed Oct. 10, 2007, all of which are hereby incorporated in their entirety. In certain embodiments, the electroporation device is a CELLECTRA® 2000 device.

In certain embodiments, administration of the nucleic acid molecule provides a statistically significant increase in overall cellular response rate in the subject relative to baseline as measured by a Lassa virus glycoprotein precursor (LASV GPC) interferon-γ (IFN-γ) ELISpot assay six weeks and/or twelve weeks after the initial dose.

In certain embodiments, administration of the nucleic acid molecule provides a statistically significant increase in overall cellular response rate in a population of subjects administered the nucleic acid molecule as measured by a Lassa virus glycoprotein precursor (LASV GPC) interferon-γ (IFN-γ) ELISpot assay relative to a population of subjects not administered the nucleic acid molecule. According to some embodiments, the increase is about 40% to about 70%.

In certain embodiments, administration of the nucleic acid molecule induces a protective immune response from Lassa virus in about 70%, about 80%, about 90%, or about 100% of a population of subjects as measured by survival following Lassa virus challenge eight weeks and/or one year following administration of the initial dose.

In certain embodiments, administration of the nucleic acid molecule induces a cellular immune response in about 65% to about 85% of a population of subjects as measured by a Lassa virus glycoprotein precursor (LASV GPC) interferon-γ (IFN-γ) ELISpot assay six weeks following administration of the initial dose. In certain embodiments, the method induces a cellular immune response in about 70% of a population of subjects as measured by a Lassa virus glycoprotein precursor (LASV GPC) interferon-γ (IFN-γ) ELISpot assay six weeks following administration of the initial dose.

In certain embodiments, administration of the nucleic acid molecule induces a cellular immune response in about 75% to about 90% of a population of subjects as measured by a Lassa virus glycoprotein precursor (LASV GPC) interferon-γ (IFN-γ) ELISpot assay twelve weeks following administration of the initial dose. In certain embodiments, the method induces a cellular immune response in about 83% of a population of subjects as measured by a Lassa virus glycoprotein precursor (LASV GPC) interferon-γ (IFN-γ) ELISpot assay twelve weeks following administration of the initial dose.

In certain embodiments, administration of the nucleic acid molecule induces a cellular immune response in about 70% to about 90% of a population of subjects as measured by a multiplex-based cytokine/chemokine assay for interleukin-2 (IL2), interferon-γ (IFN-γ), tumor necrosis factor-α (TNF-α), IFN-γ-induced protein-10 (IP10), and monokine-induced-by-IFN-γ (MIG) six weeks following administration of the initial dose. In certain embodiments, the method induces a cellular immune response in about 80% of a population of subjects as measured by a multiplex-based cytokine/chemokine assay for interleukin-2 (IL2), interferon-γ (IFN-γ), tumor necrosis factor-α (TNF-α), IFN-γ-induced protein-10 (IP10), and monokine-induced-by-IFN-γ (MIG) six weeks following administration of the initial dose.

In certain embodiments, administration of the nucleic acid molecule induces a cellular immune response in about 70% to about 90% of a population of subjects as measured by a multiplex-based cytokine/chemokine assay for interleukin-2 (IL2), interferon-γ (IFN-γ), tumor necrosis factor-α (TNF-α), IFN-γ-induced protein-10 (IP10), and monokine-induced-by-IFN-γ (MIG) twelve weeks following administration of the initial dose. In certain embodiments, the method induces a cellular immune response in about 80% of a population of subjects as measured by a multiplex-based cytokine/chemokine assay for interleukin-2 (IL2), interferon-γ (IFN-γ), tumor necrosis factor-α (TNF-α), IFN-γ-induced protein-10 (IP10), and monokine-induced-by-IFN-γ (MIG) twelve weeks following administration of the initial dose.

In certain embodiments, administration of the nucleic acid molecule results in no sensoneuronal hearing loss in the subject.

### EXAMPLES

The examples and embodiments described herein are for illustrative purposes only and various modifications or changes suggested to persons skilled in the art are to be included within the spirit and purview of this application and scope of the appended claims.

### Example 1: INO-4500 Immunogenicity and Protective Efficacy in a Dose-Ranging NHP Challenge Study

INO-4500 was evaluated in a dose-ranging study to determine protective efficacy of lower vaccine doses in the nonhuman primate (NHP) lethal LASV challenge model. Cynomolgus NHPs (n of 6/group) received two immunizations spaced four weeks apart of either 0.1 mg, 1 mg, or 2 mg of INO-4500 administered by intradermal (ID) injection followed by electroporation (EP) (ID-EP) or were not immunized as infection controls. Humoral (GPC ELISA and pseudovirus neutralization) and T cell (IFNγ ELISpot) responses were at week 0 (pre-challenge) and two weeks post each immunization (week 2 and week 6). At four weeks post second immunization (week 8), NHPs were challenged via intramuscular (IM) injection with 1,000 pfu of LASV (Josiah strain) and monitored daily for survival and clinical signs of disease. Blood samples collected from the NHPs were monitored for neutralizing antibodies, complete blood counts (CBC) and blood chemistries and the animals were observed for signs of disease progression. All surviving animals were euthanized at 38 days post-infection for necropsy and gross pathology.

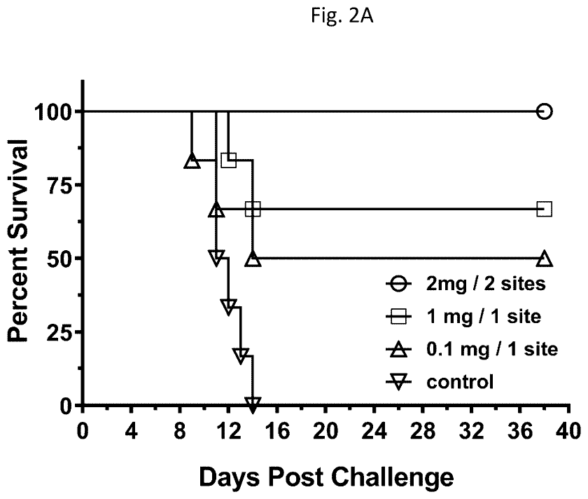

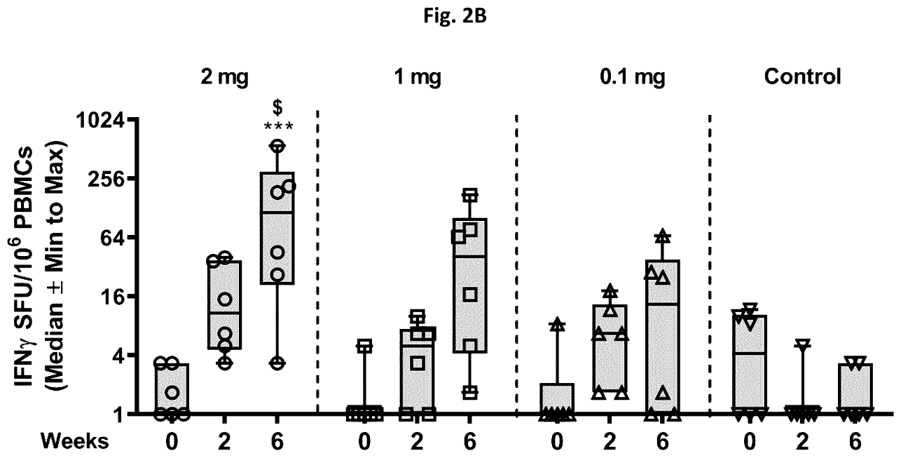

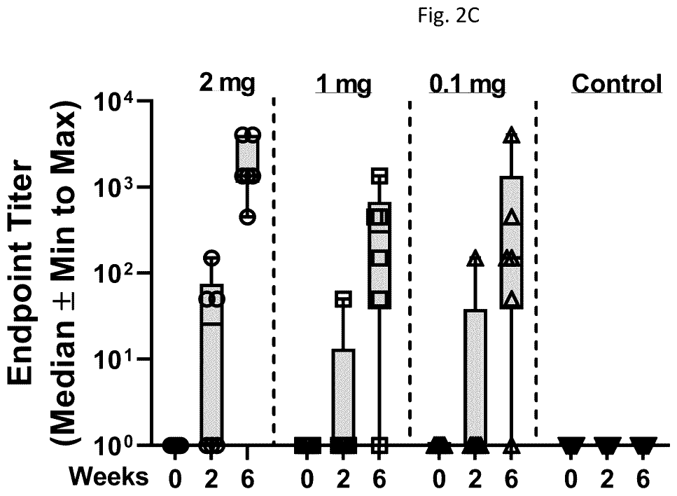

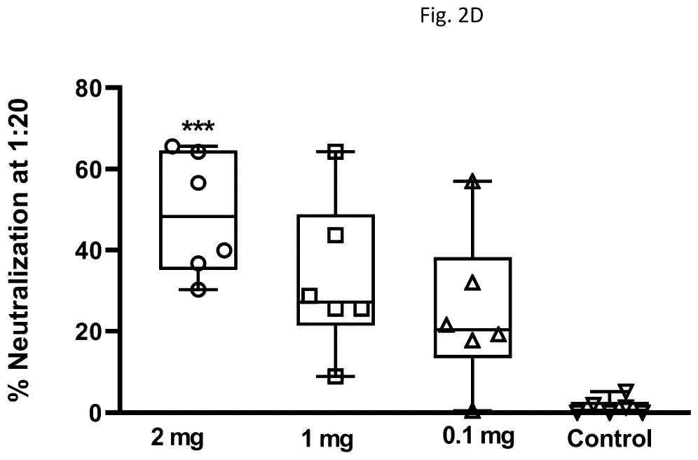

In all control animals, onset of clinical signs of disease occurred between days 10 to 12 post-exposure and all (6/6) met the criteria for euthanasia during the hemorrhagic phase at days 11 to 14 post infection. In contrast, 3/6, 4/6, and 6/6 NHPs immunized with 0.1 mg, 1 mg or 2 mg of INO-4500, respectively, survived lethal LASV challenge, demonstrating the protective efficacy over a range of INO-4500 vaccine doses (FIG. 2A). Gross necropsy analysis demonstrated all control animals euthanized presented clear pathological findings. All vaccinated animals that survived the challenge demonstrated normal gross necropsy analysis. In addition, no sensorineural hearing loss was detected in surviving vaccinated NHPs (38 days post-challenge). Magnitude of cellular responses (IFNγ ELISpot) were INO-4500 dose-related, and detected above baseline at WEEK 6 for 5/6 NHPs each in the 2 mg and 1 mg dose groups and 3/6 NHPs in the 0.1 mg dose groups (FIG. 2B). Sera IgG binding titers against clade IV LASV GPC were measured by ELISA at WEEKs 0, 2 and 6 (FIG. 2C). Binding antibody levels were dose-related, and were detected in 6/6, 5/6, and 5/6 NHPs in the 2, 1, and 0.1 mg dose groups, respectively. Sera neutralizing antibody responses against clade IV LASV were measured by pseudovirus assay at WEEK 6 (FIG. 2D). Sera neutralizing activity was dose-related, and was detected 6/6, 5/6, and 5/6 NHPs for the 2, 1, and 0.1 mg dose groups, respectively. Neutralizing activity was not detected for any control non-vaccinated NHPs. Of the 5/18 INO-4500 immunized NHPs that developed hemorrhagic disease and were euthanized, two showed no antibody or T cell response (#7367, 7381), one showed weaker antibody response with no T cell response (#7368), and one showed weaker antibody response with modest T cell response (#7375). Although the study was not designed for statistical power to determine potential correlates or protection, the data supports a trend of increasing antibody and/or T cell responses associated with INO-4500 mediated protection from lethal LASV challenge.

Durability of INO-4500 Protective Efficacy. The long-term protective efficacy of INO-4500 was evaluated in the NHP LASV challenge model. Cynomolgus NHPs (n of 6/group) received two immunizations spaced four weeks apart of either 1 or 2 mg of INO-4500 administered by ID injection followed by EP. Non-immunized NHPs (n of 6) served as infection controls. Humoral and T cell responses were assessed as in the study above prior to immunization (week 0), and week 2 and week 6, and Months 4, 6, 9 and 12 post start of immunization. At Month 12 (week 59), NHPs were challenged via intramuscular injection with 1,000 pfu of LASV (Josiah strain) and monitored daily for survival and clinical signs of disease.

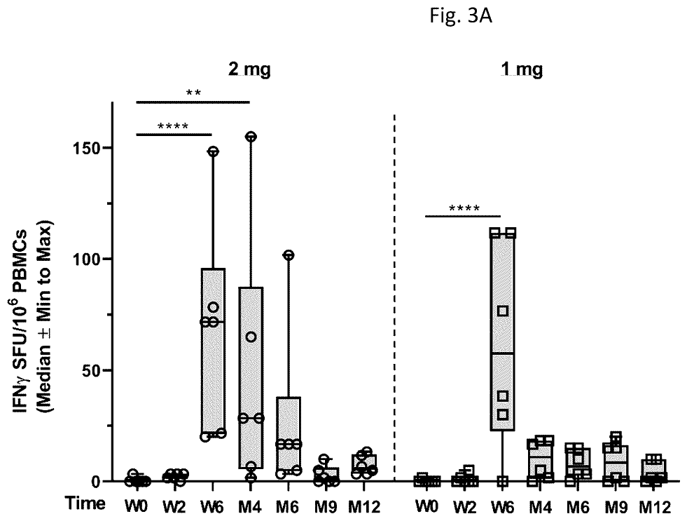

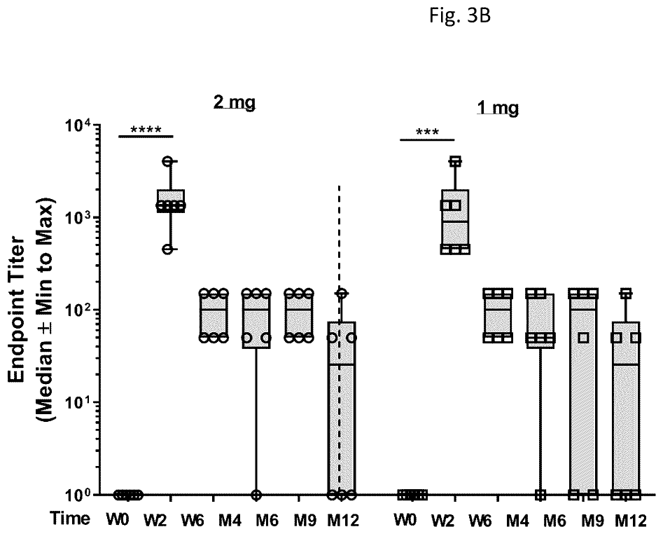

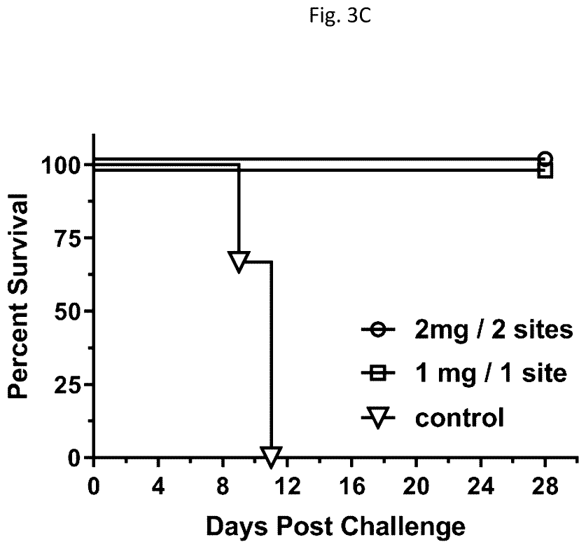

All unvaccinated control animals (6/6) were either euthanized or succumbed to disease during the hemorrhagic phase between days 9 and 12 post infection (FIG. 3C). In contrast, all 12/12 NHPs immunized with either 1 or 2 mg INO-4500 survived lethal LASV challenge. T cell responses (IFNγ ELISpot) above baseline were detected in 6/6 and 5/6 INO-4500 immunized NHPs in the 2 mg and 1 mg dose groups, respectively. Peak cellular responses for both dose groups were observed at week 6, which declined but remained elevated above baseline for most immunized NHPs through Months 4 and 6 before waning by Months 9 and 12 (FIG. 3A). All 12/12 INO-4500 immunized NHPs seroconverted (clade IV LASV GPC ELISA), with peak binding antibody titers for both dose groups were observed at WEEK 6, which declined that remained elevated above baseline for most immunized NHPs Month 4 through Month 9 (FIG. 3B). Binding antibody titers were still detectable for 3/6 NHPs in each dose group at 12 months post-immunization. These findings suggest induction of a memory response following immunization with INO-4500, resulting in durable protective efficacy against lethal LASV infection.

### Example 2: Protective Efficacy of One or Two INO-4500 Immunizations in an NHP Challenge Study

INO-4500 was evaluated as a single dose regimen to define the minimum requirements for protection. Cynomolgus NHPs (n of 6/group) received either one (WEEK 0) or two (WEEKs 0 and 4) immunizations with 2 mg of INO-4500 administered ID followed by EP. Non-immunized NHPs (n of 6) served as infection controls. Humoral and T cell immune responses were assessed at WEEK0 (pre-challenge), and WEEKs 2, 4 and 6. NHPs were challenged at WEEK 8 (eight weeks after single immunization and four weeks after two immunizations) via IM injection with 1,000 pfu of LASV (Josiah strain) and monitored daily for survival and clinical signs of disease.

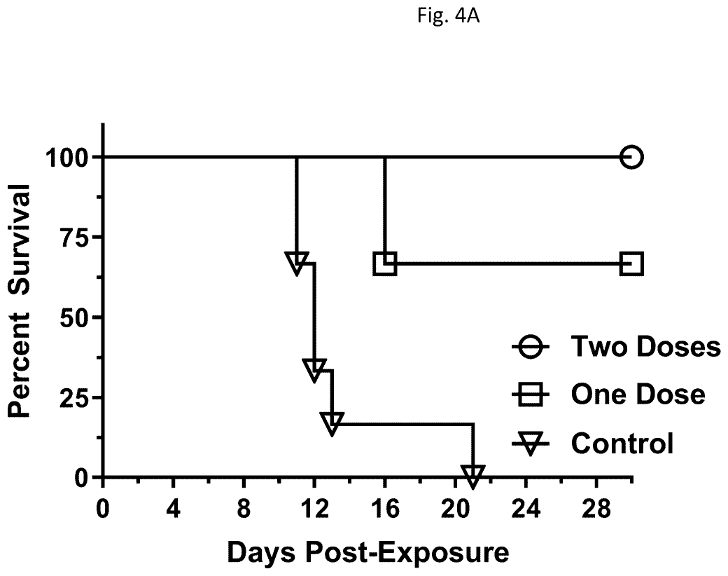

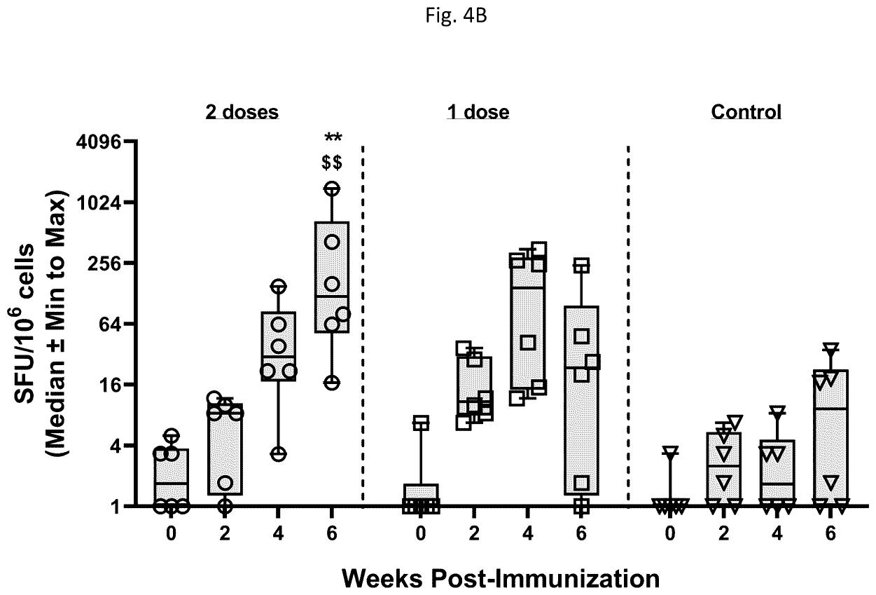

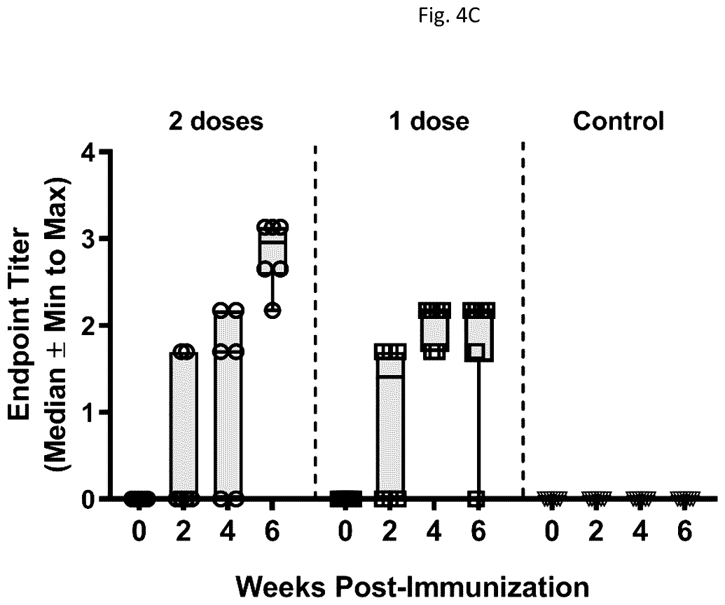

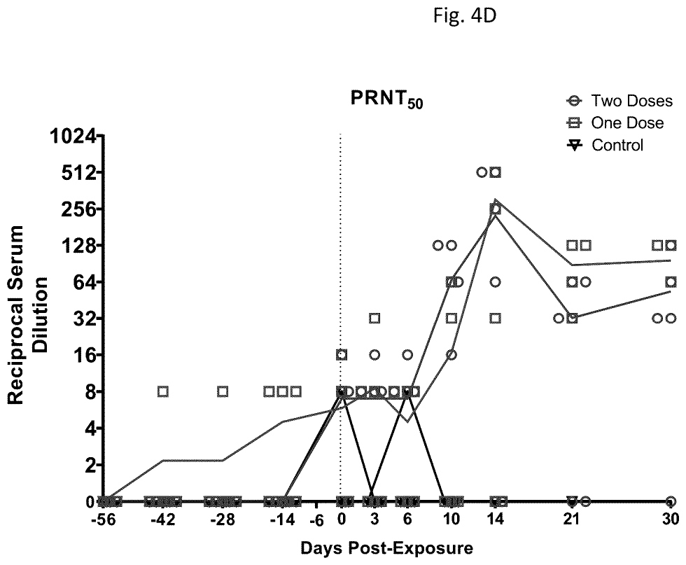

In all control animals, onset of clinical signs of disease occurred between days 10 to 12 post-exposure and all (6/6) met the criteria for euthanasia during the hemorrhagic phase at days 11 to 14 post infection (FIG. 4A). In contrast, 6/6 and 4/6 NHPs immunized with two or one dose of INO-4500, respectively, survived lethal LASV challenge, demonstrating the protective efficacy of INO-4500 even when used at a single 2 mg dose. Serum viremia was assessed post-challenge (not shown). All 6/6 control animals developed viremia that peaked between days 10 to 13 post-challenge (average 7.9 log pfu/mL). Viremia was undetectable for 5/6 NHPs that received two 2 mg INO-4500 immunizations, and 2.5 logs lower for 1/6 as compared to control infected NHPs. While viremia was detected in all 6/6 NHPs that received only one INO-4500 immunization, average peak viral loads were 3.5 logs lower than control animals. This data suggests that single immunization of INO-4500 could limit viral Lassa viral replication. T cell responses (ELISpot) at week 6 were detected in all 6/6 NHPs in the two-dose group, and in 4/6 in the one-dose group (FIG. 4B). Sera IgG binding titers against clade IV LASV GPC were measured by ELISA at weeks 0, 2, and 6 post-initial immunization with INO-4500. A 100% seroconversion rate was observed for both vaccinated groups (6/6, LASV GPC ELISA), with the one-dose group showing 100% seroconversion at week 4 post-dose and the two-dose group at week 6, or 2 weeks after the second dose of INO-4500 (FIG. 4C). None of the animals in the control group showed binding responses to LASV GPC. Pre- and post-challenge levels of LASV neutralizing antibodies were measured by PRNT assay (FIG. 4D). Neutralizing antibody levels were low to undetectable for all study NHPs prior to Lassa challenge. After challenge, neutralizing responses rapidly increased for both immunization groups with neutralizing tiers ≥64 detected for 4/6 and 6/6 NHPs receiving one or two INO-4500 immunizations, respectively. No neutralizing activity was detected in the control group, or for the 2 non-surviving NHPs in the one-dose group. This data would indicate the presence of an antigen-specific memory B cell population following INO-4500 immunization that can be rapidly reactivated upon Lassa challenge.

### Example 3: Cross-Reactive Immunity of INO-4500 Against Heterologous LASV Strains

The INO-4500 study described above evaluated the immunogenicity against LASV GPC antigens of the matched clade IV Josiah strain. INO-4500 was therefore evaluated for its ability to induce cross-reactive immune responses against additional LASV strains. Five NHPs previously immunized with INO-4500 were evaluated for cross-reactive immunogenicity against heterologous LASV strains. These NHPs received a booster immunization of 2 mg of INO-4500 administered intradermally followed by electroporation (EP) (ID-EP) at week 37 (˜9 months after start of a primary immunization series consisting of a three 2 mg dose regimen done at week 0, week 4 and week 8) for collection of PBMCs to evaluate LASV cross-clade cellular responses pre- (week 37) and post- (week 39) boost. Sera samples collected during primary immunization series (weeks 0, 2, 6 and 10), pre-boost (week 37) and post-boost (week 39) were evaluated for cross-clade binding antibodies and neutralizing activity.

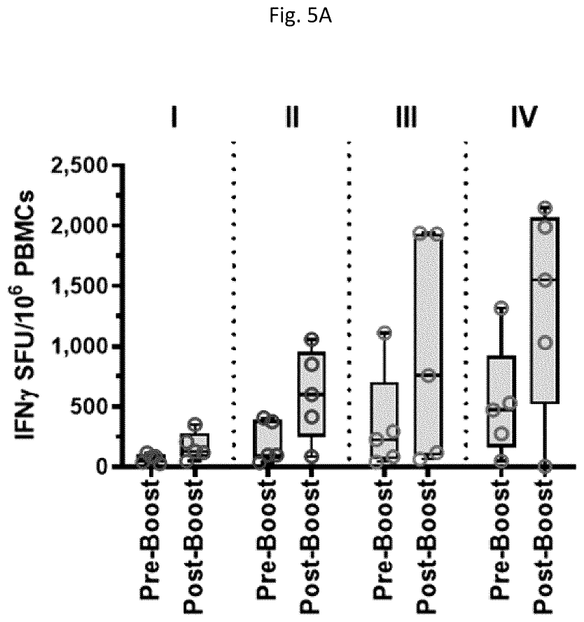

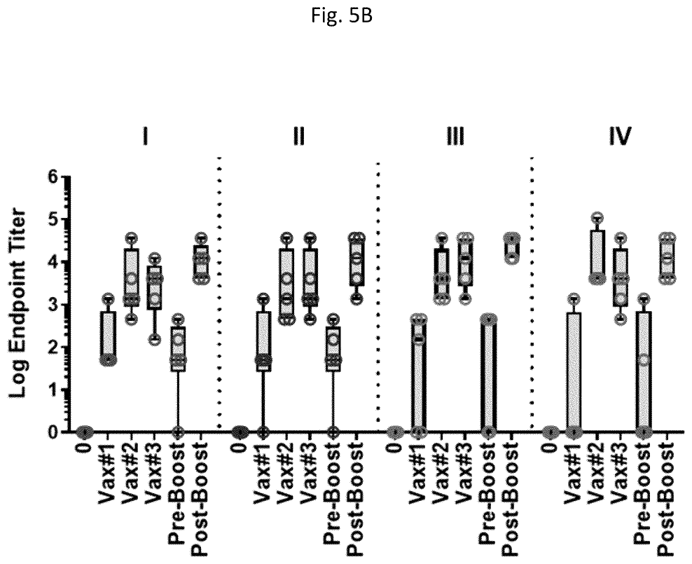

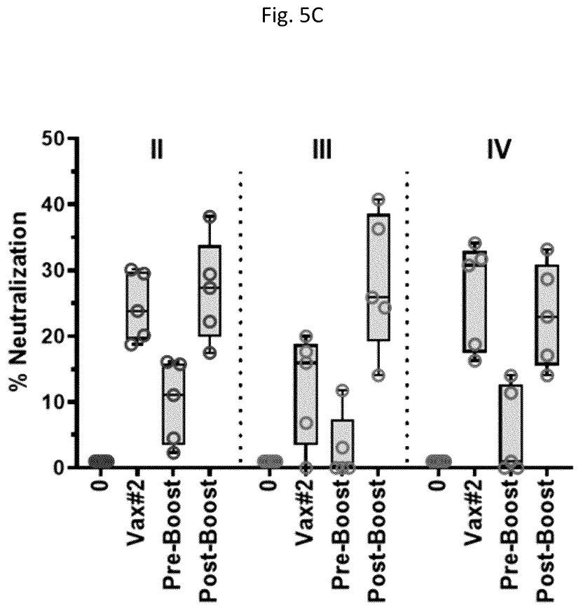

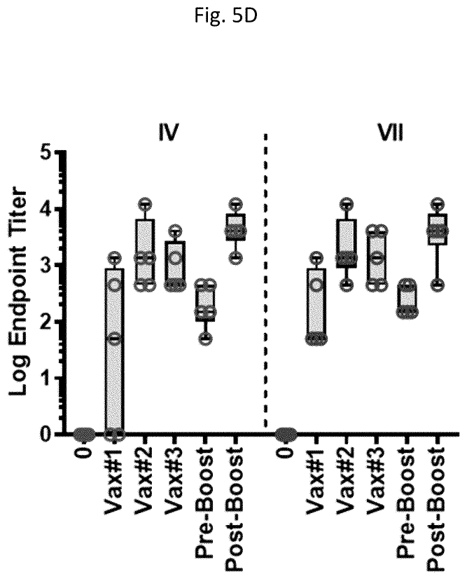

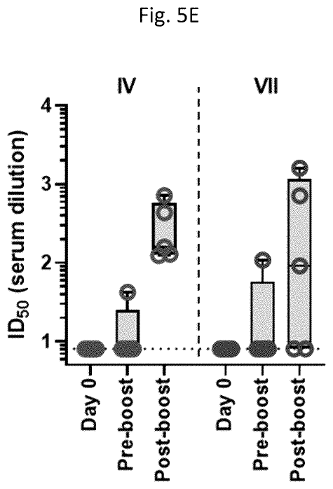

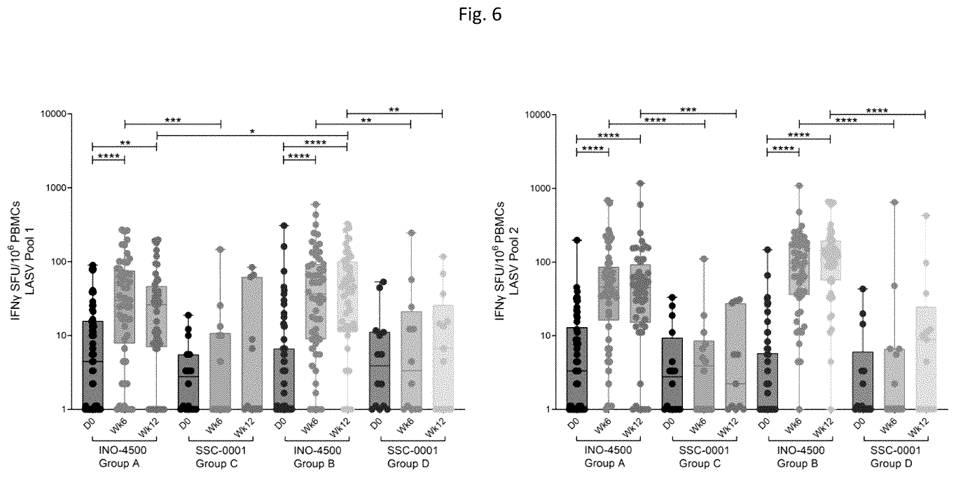

T cell based immune responses against clade I-IV LASV were detected by IFNγ ELISpot in all (5/5) NHPs pre-boost and were increased following booster immunization at comparable levels across clades (FIG. 5A). PBMCs were not available at time of Clade VII evaluation. Sera IgG binding titers against clade I-IV and VII LASV GPCs were detected by ELISA in some animals as early as week 2, with a 100% seroconversion rate (5/5) against GPCs for all LASV clades by week 6 (FIGS. 5B, 5D). Binding antibody titers were comparable across all LASV clades. Binding titers decreased by the pre-boost timepoint at ˜9 months post immunization. However, antibody titers against all clades increased following boost comparable to week 6 levels, indicating induction of a memory B cell response against heterologous LASV clades.

To evaluate induction of cross-clade neutralizing antibody responses to INO-4500 immunization, a pseudovirus neutralization assay using pseudoviruses expressing a GFP reporter gene and displaying GPCs of LASV clades II, III or IV (FIG. 5C) was employed. Sera neutralizing activity against all clades tested were detected in 5/5 animals after two immunizations with INO-4500 (week 6). Neutralizing activity decreased by the pre-boost timepoint at ˜9 months post immunization, then increased for all clades following boost comparable to week 6 levels, consistent with kinetics observed for IgG binding titers. We further generated pseudovirus expressing LASV GPC corresponding to Clade I and Clade VII (Benin) carrying a luciferase reporter gene (FIG. 5E). Neutralizing responses were detected in 5/5 NHPs against Clade IV pseudovirus, and in the majority of NHPs (3/5) against Clade VII and I pseudoviruses.

### Example 4: Study to Evaluate the Safety, Tolerability and Immunogenicity of INO-4500 in Healthy Volunteers (LSV-001) (Clinicaltrials_gov Identifier NCT03805984)

This study is a Phase 1, randomized, double-blinded, and placebo-controlled study to evaluate the safety, tolerability and immunological profile of INO-4500 administered by intradermal (ID) injection followed by electroporation (EP). This was the first-in-human study in healthy adult volunteers in the United States. A total of 60 subjects were enrolled.

Subjects received either 1 mg or 2 mg of INO-4500 (or corresponding placebo [SSC-0001] regimen) at week 0 and week 4 as summarized in Table 1. INO-4500 and SSC-0001 were administered in ˜0.1 mL dose volume. Blood was drawn from all subjects for antibody and T-cell responses at baseline (Screening and pre-dose at week 0) and at all subsequent study visits.

TABLE 1
LSV-001 Study Design

| | | | | Number of | | Total |
|---|---|---|---|---|---|---|
| Study | | | | Injections + | INO-4500 | Dose of |
| Dose | | | Dosing | EP per Dosing | (mg) per | INO-4500 |
| Group | Number of Subjects | | Weeks | Visit | injection | (mg) |
| A | 6 | (4 active, 2 placebo) | 0, 4 | 1 | 1 | 2 |
| B | 54 | (36 active, 18 placebo) | 0, 4 | 2 | 1^{a} | 4 |

| | |
|---|---|
| Total | 60 |

^{a}INO-4500 will be injected ID followed by EP in two different acceptable locations at each dosing visit.

Trial Treatment:
Group A—One 1 mg injection of INO-4500 or SSC-0001 ID injection followed by EP will be administered at Day 0 and Week 4 (+5 days)
Group B—Two 1 mg injections of INO-4500 or SSC-0001 ID injection followed by EP will be administered at Day 0 and Week 4 (+5 days)

Investigational Product: INO-4500 is the active investigational product used in this study. The INO-4500 drug product contains 10 mg/mL pGX9808 in 1×SSC buffer (150 mM sodium chloride and 15 mM sodium citrate, pH 7.0). A volume of 0.4 mL will be filled into 2-mL glass vials that are fitted with rubber stoppers and sealed aluminum caps.

Study Population:

Inclusion Criteria: Subjects were eligible for inclusion in the clinical trial if they met all of the following criteria: able to provide informed consent; adult age 18 to 50 years, inclusive; judged to be healthy on the basis of medical history, physical examination and vital signs performed at Screening; able and willing to comply with all study procedures; screening laboratory results within normal limits for testing laboratory or deemed not clinically significant; negative serological tests for Hepatitis B surface antigen (HBsAg), Hepatitis C antibody and Human Immunodeficiency Virus (HIV) antibody or rapid test at Screening; screening ECG deemed as having no clinically significant findings; must meet one of the following criteria with respect to reproductive capacity: women who are post-menopausal as defined by spontaneous amenorrhea for ≥12 months, surgically sterile or have a partner who is sterile, use of medically effective contraception, abstinence.

Exclusion Criteria: Subjects were to be excluded from participation in this clinical trial if they met any of the following criteria: pregnant or breastfeeding, or intending to become pregnant or father children within the projected duration of the trial starting with the Screening visit until 1 month following last dose; positive serum pregnancy test during Screening or positive urine pregnancy test prior to dosing; currently participating in or has participated in a study with an investigational product with 30 days preceding Day 0; previous receipt of an investigational vaccine product for prevention of Lassa Fever; audiometry testing that demonstrates a hearing level threshold of 30 dB or greater for any frequency tested between 250 Hz-8000 Hz; fewer than two acceptable sites available for ID injection and EP considering the deltoid and anterolateral quadriceps muscles; prisoner or subjects who are compulsorily detained; current or anticipated concomitant immunosuppressive therapy (excluding inhaled, topical skin and/or eye drop-containing corticosteroids); reported active drug or alcohol or substance abuse or dependence; recent (within 6 months) or planned travel to Lassa-endemic region.

Study Objectives and Endpoints:

The Primary Objective of the study was to evaluate the tolerability and safety of INO-4500 administered by ID injection followed by EP in healthy adult volunteers. The Primary Safety Endpoints included incidence of adverse events classified by systemic organ class (SOC), preferred term (PT), severity and relationship to investigational product; administration (e.g., injection) site reactions (described by frequency and severity); and incidence of adverse events of special interest.

The Secondary Objective of the study was to evaluate the cellular and humoral immune response to INO-4500 administered by ID injection followed by EP. The Secondary Endpoints included antigen specific binding antibody titers; LASV neutralizing antibodies; and antigen specific cellular immune response by IFN-γ ELISpot and/or flow cytometry assays.

The study's Exploratory Objective was to evaluate the expanded immunological profile by assessing both T and B cell immune responses. The Exploratory Endpoint was an expanded immunological profile to include (but not limited to) additional assessment of T and B cell numbers and T and B cell molecular changes by measuring immunologic proteins and mRNA levels of genes of interest at all weeks as determined by sample availability.

### Study Assessments:

Immunogenicity Assessment: Immunology samples are collected at Screening, Day 0, Week 2, Week 4, Week 6, Week 12, Week 24 and Week 48. Determination of analysis of collected samples for immunological endpoints will be determined on an ongoing basis throughout the study. The T and B cell immune responses to INO-4500 will be measured using assays that include but are not limited to ELISA, neutralization, assessment of immunological gene expression, assessment of immunological protein expression, flow cytometry and ELISPOT.

Safety Assessment: Adverse events are collected at every visit and safety samples are drawn at Screening, Week 2, Week 6, and Week 48. The safety of INO-4500 will be measured and graded in accordance with the “Toxicity Grading Scale for Healthy Adult and Adolescent Volunteers Enrolled in Preventive Vaccine Clinical Trials”, issued September 2007 (Appendix A).

Hearing assessment will be performed on subjects using pure-tone audiometry at Screening and study discharge (Week 48 or any other study discontinuation visit), or as clinically indicated. Pure-tone audiometry evaluates if hearing is within normal-to-mild limits by checking hearing capabilities at certain frequencies (e.g., 250, 500, 1000, 2000, 3000, 4000, 6000 and/or 8000 Hz). If hearing falls outside of normal-to-mild limits, bone conduction or tympanometry test may be completed to determine the specific type of hearing loss. In addition, the following information should be obtained: recent ear pain or drainage, injuries to the ear or other head injuries, any loud noise exposure (occupational, blast, firearm, etc), new or change in ringing/buzzing/hissing sounds, any known issues with ear wax, chemotherapy, or hospitalization requiring IV antibiotics. The hearing assessment will include hearing level (dB HL) at the measured frequencies.

### Statistical Analyses:

Analysis populations will be:

The modified intention to treat (mITT) population includes all subjects who receive at least one dose of the INO-4500 or SSC-0001. Subjects in this sample will be analyzed by their original assigned dose of INO-4500 or SSC-0001. The mITT population will be used to analyze secondary and exploratory immunological endpoints.

The safety analysis population includes all subjects who receive at least one dose of INO-4500 or SSC-0001 administered by ID injection. Subjects for this population will be grouped according to the dose of INO-4500 or SSC-0001 that they received. This population will be used for all safety analyses in the study.

Analysis of Primary Safety Endpoints. The primary analyses for this trial are safety analyses of Treatment Emergent Adverse Events (TEAE), administration site reactions and clinically significant changes in safety laboratory parameters from baseline. TEAEs are defined for this trial as any AEs/SAEs that occur on or after Day 0 up to 30 days after each dose of IP administration. All TEAEs will be summarized by frequency, percentage and associated 95% Clopper-Pearson confidence interval, and the difference in the percentage between those who received INO-4500 and SSC-0001 and associated 95% exact confidence interval within each study group. The frequencies will also be presented separately by dose number and will be depicted by system order class and preferred term. Additional frequencies will be presented with respect to maximum severity and relationship to IP. Multiple occurrences of the same AE in a single subject will be counted only once following a worst-case approach with respect to severity and relationship to IP. All serious TEAEs will also be summarized as above. AE duration will be calculated as AE stop date−AE start date+1 day. AEs and SAEs that are not TEAEs or serious TEAEs will be presented in listings. All of these primary safety analyses will be conducted on the subjects in the safety population.

Analysis of Secondary Immunology Endpoints. Antigen specific binding antibody titers, LASV neutralizing antibody titers, and antigen specific cellular immune responses will be analyzed descriptively by dose groups within Study Groups A and B at Day 0 and up to Week 6. Binding antibody titer fold increases from day 0 will be analyzed by dose group within each study group using the geometric mean and associated 95% confidence intervals up to Week 6. Increases from Day 0 up to Week 6 for neutralizing antibody titer and antigen specific cellular immune responses will be analyzed by dose group within each study group using medians, inter-quartile range and associated 95% confidence intervals.

Exploratory Analyses. Antigen specific binding antibody titers, LASV neutralizing antibody titers, and antigen specific cellular immune responses may be analyzed at Weeks 48 in the same manner as described for the respective secondary analyses. T and B post baseline cell number fold-changes will be analyzed descriptively by dose group within each study group using the geometric mean and associated 95% confidence intervals.

### Study Results:

Immunogenicity Results: Cellular response rates as measured by LASV GPC IFN-γ ELISpot revealed a 44.4% overall ELISpot response rate for Group B (INO-4500 2 mg) compared with 0% response rate for the Placebo group. IFN-γ ELISpot levels were significantly greater at week 6 and week 12 for subjects in Group B as compared to both baseline and Placebo. Humoral response rates as measured by LASV GPC binding IgG ELISA were 9.4% for the 2 mg INO-4500 cohort, and 0% for the 1 mg INO-4500 and Placebo cohorts (Table 2).

TABLE 2
LSV-001: Unblinded Immunology Summary Table

| | | | INO-4500 + EP | | SSC-0001 + EP | |
|---|---|---|---|---|---|---|
| | | | Group A | Group B | Group A | Group B |
| Assay | Analysis | Statistics | (N = 4) | (N = 36) | (N = 2) | (N = 18) |
| ELISpot | GPC Pool 1 | n/N (%) | 0/4 (0.0) | 8/36 (22.2) | 0/1 (0.0) | 0/18 (0.0) |
| | | 95% CI | 0.0, 60.2 | 10.1, 39.2 | 0.0, 97.5 | 0.0, 18.5 |
| ELISpot | GPC Pool 2 | n/N (%) | 0/4 (0.0) | 13/36 (36.1) | 0/1 (0.0) | 0/18 (0.0) |
| | | 95% CI | 0.0, 60.2 | 20.8, 53.8 | 0.0, 97.5 | 0.0, 18.5 |
| ELISpot | Overall* | n/N (%) | 0/4 (0.0) | 16/36 (44.4) | 0/1 (0.0) | 0/18 (0.0) |
| | | 95% CI | 0.0, 60.2 | 27.9, 61.9 | 0.0, 97.5 | 0.0, 18.5 |
| ELISA | Binding | n/N (%) | 0/4 (0.0) | 3/32 (9.4) | 0/1 (0.0) | 0/18 (0.0) |
| | | 95% CI | 0.0, 60.2 | 1.98, 25.0 | 0.0, 97.5 | 0.0, 18.5 |

*Overall ELISpot responses for any subject is defined as a response to any GPC pool

Safety Results: INO-4500 (at a dose of 1 mg or 2 mg) was safe and well tolerated when administered ID with EP. A total of 22 subjects (36.7%) experienced at least one treatment-emergent adverse event (TEAE). Most TEAEs were Grade 1 or 2 in severity. The only Grade 3 TEAE was increased blood glucose experienced by one (1.7%) subject. No subjects experienced a Grade >3 TEAE, and there were no treatment-emergent SAEs. There were no TEAEs that led to treatment discontinuation or study discontinuation. There were no cases of attributable hearing loss. A total of four (4) subjects (6.7%) experienced at least one (1) TEAE that was assessed as treatment related by the Investigator.

### Example 5: Dose-Ranging Study to Evaluate the Safety, Tolerability and Immunogenicity of INO-4500 in Combination with Electroporation in Healthy Volunteers in Ghana (LSV-002) (Clinicaltrials_Gov Identifier NCT04093076)

This is a Phase Tb, randomized and blinded, placebo-controlled trial to evaluate the safety, tolerability and immunological profile of INO-4500 administered by intradermal (ID) injection followed by electroporation (EP) in healthy adult volunteers. The primary objectives of this trial are to evaluate the tolerability and safety, as well as cellular and humoral immune response, of INO-4500 administered by ID injection followed by EP in healthy adult volunteers in accordance with the regimens outlined in Table 3.

Approximately 220 healthy volunteers will be evaluated across two dose regimens and two corresponding placebo regimens. Participants will be randomized to receive a two-dose series of either active investigational product (INO-4500; 176 participants) or placebo (SSC-0001; 44 participants).

TABLE 3
Study Dosing Regimens

| | | | | | | Total |
|---|---|---|---|---|---|---|
| | | | | Number of | | Dose |
| | | Number | | Injections + | INO- | of |
| | | of | | EP per | 4500 | INO- |
| | | Partic- | Dosing | Dosing | (mg) per | 4500 |
| Dosing Regimens | | ipants | Weeks | Visit | Injection | (mg) |
| Active | A | 88 | 0, 4 | 1 | 1 | 2 |
| (INO-4500) | B | 88 | 0, 4 | 2^{a} | 1 + 1 (2) | 4 |
| Placebo | C | 22 | 0, 4 | 1 | 0 | 0 |
| (SSC-0001) | D | 22 | 0, 4 | 2^{a} | 0 | 0 |

| | |
|---|---|
| Total | 220 |

^{a}INO-4500 or SSC-0001 will be injected ID followed by EP in an acceptable location on two different limbs at each dosing visit.
INO-4500 or SSC-0001 will be administered in ~0.1 mL dose volume followed immediately by EP in the participant's deltoid or anterolateral quadriceps.

Trial Treatment:

Active Dosing Regimens:
Regimen A—One 1 mg ID injection of INO-4500 followed by EP will be administered at Day 0 and Week 4 (±5 days)
Regimen B—Two 1 mg ID injections (different limbs) of INO-4500 followed by EP will be administered at Day 0 and Week 4 (±5 days)

Placebo Dosing Regimens:
Regimen C—One ID injection of SSC-0001 followed by EP will be administered at Day 0 and Week 4 (±5 days)
Regimen D—Two ID injections (different limbs) of SSC-0001 followed by EP will be administered at Day 0 and Week 4 (±5 days)

### Study Objectives and Endpoints:

The Primary Objectives of the study include evaluation of the tolerability and safety of INO-4500 administered by ID injection followed by EP in healthy adult volunteers; and evaluation of the cellular and humoral immune response to INO-4500 administered by ID injection followed by EP. Primary Safety Endpoints are incidence of adverse events classified by systemic organ class (SOC), preferred term (PT), severity and relationship to investigational product; administration (e.g., injection) site reactions (described by frequency and severity); and incidence of adverse events of special interest. The Primary Immunogenicity Endpoints are LASV-antigen specific antibodies by binding and/or neutralization assays and antigen specific cellular immune response by IFN-γ ELISpot and/or flow cytometry assays.

The Exploratory Objective of the study is to evaluate the expanded immunological profile by assessing both T and B cell immune responses. The Exploratory Endpoint is an expanded immunological profile which may include (but not limited to) additional assessment of T and B cell numbers and T and B cell molecular changes by measuring immunologic proteins and mRNA levels of genes of interest at all weeks as determined by sample availability.

Study Population:

Inclusion Criteria: Subjects were eligible for inclusion in the clinical trial if they met all of the following criteria: able to provide informed consent; adult age 18 to 50 years, inclusive; judged to be healthy on the basis of medical history, physical examination and vital signs performed at Screening; able and willing to comply with all study procedures; screening laboratory results within normal limits for testing laboratory or deemed not clinically significant; negative serological tests for Hepatitis B surface antigen (HBsAg), Hepatitis C antibody and Human Immunodeficiency Virus (HIV) antibody or rapid test at Screening; screening ECG deemed as having no clinically significant findings (e.g., Wolff-Parkinson-White syndrome); meet one of the following criteria with respect to reproductive capacity: women who are post-menopausal as defined by spontaneous amenorrhea for ≥12 months, surgically sterile or have a partner who is sterile, or use of medically effective contraception when used consistently and correctly from Screening until three (3) months following last dose.

Exclusion Criteria: Subjects were to be excluded from participation in this clinical trial if they met any of the following criteria: pregnant or breastfeeding, or intending to become pregnant within the projected duration of the trial starting with the Screening visit until three (3) months following last dose; positive serum pregnancy test during Screening or positive urine pregnancy test prior to any dosing; currently participating in or has participated in a study with an investigational product with 30 days preceding Day 0; previous receipt of an investigational vaccine product for prevention of Lassa Fever; audiometry testing that demonstrates a hearing level threshold greater than 30 dB for any frequency tested between 500 Hz-8000 Hz; fewer than two acceptable sites available for ID injection and EP considering the deltoid and anterolateral quadriceps muscles; prisoner or participants who are compulsorily detained; current or anticipated concomitant immunosuppressive therapy (excluding inhaled, topical skin and/or eye drop-containing corticosteroids); reported active drug or alcohol or substance abuse or dependence; or fever with or without cough or any other concurrent illness contraindicated to clinical trial participation.

Investigational Product:

INO-4500 is the active investigational product used in this study. The INO-4500 drug product contains 10 mg/mL pGX9808 in 1×SSC buffer (150 mM sodium chloride and 15 mM sodium citrate, pH 7.0). A volume of 0.4 mL will be filled into 2-mL glass vials that are fitted with rubber stoppers and sealed aluminum caps.

Placebo:

Sterile SSC Buffer (SSC-0001), which contains 150 mM sodium chloride and 15 mM sodium citrate, pH 7.0 in 10-mL glass vials, stoppered, and sealed with aluminum caps, will be used as the placebo.

Trial Procedures:

Participants in Dosing Regimens A and C (Groups A and C, respectively) will receive one (1) injection of INO-4500 or SSC 0001 in volume of ˜0.1 mL by ID injection above an acceptable location for injection. The injection site is assessed for IP leakage and followed immediately by EP. Participants in Regimens B and D (Groups B and D, respectively) will receive one (1) ID injection of INO 4500 or SSC-0001 above each of two (2) acceptable locations for injection (on different limbs), each in a volume of ˜0.1 mL. The injection sites are assessed for IP leakage and followed immediately by EP.

### Study Assessments:

Safety Assessment: Adverse events are collected at every visit until Week 24 and safety samples are drawn at Screening, Week 2, Week 6, and Week 48. Adverse events of special interest, SAE and related AEs are collected until the end of the study. The safety of INO-4500 and SSC-0001 will be measured and graded in accordance with the “Toxicity Grading Scale for Healthy Adult and Adolescent Volunteers Enrolled in Preventive Vaccine Clinical Trials”, issued September 2007 (Appendix B).

Hearing Assessment. Hearing assessment/audiometry evaluation will be performed on participants using pure-tone audiometry at Screening, Week 4, prior to IP administration and study discharge (Week 48 or any other study discontinuation visit). Pure-tone audiometry evaluates if hearing is within normal-to-mild limits by evaluating hearing capabilities at certain frequencies: 500, 1000, 2000, 4000, 6000 and 8000 Hz. If hearing falls outside of normal-to-mild limits, bone conduction testing may be completed to determine the specific type of hearing loss. The hearing assessment will include hearing level (dB HL) at the measured frequencies.

Table 4 below defines degrees of hearing loss based on pure-tone audiogram thresholds.

TABLE 4
Classification of Hearing Loss by Severity
Degree of Hearing Loss (HL) (pure tone threshold
on hearing level audiogram)

| | | |
|---|---|---|
| 0-15 | dB | Within normal limits |
| 16-25 | dB | Slight HL |
| 26-40 | dB | Mild HL |
| 41-55 | dB | Moderate HL |
| 56-70 | dB | Moderate to severe (or moderately severe) HL |
| 71-90 | dB | Severe HL |
| 91+ | dB | Profound HL |

Liu et al. Sensorineural hearing loss (SNHL) as an adverse event following immunization (AEFI): Case definition and guidelines for data collection, analysis, and presentation of immunization safety data. Vaccine. 2020; 7.

Immunogenicity Assessment: Immunology samples are collected at Screening and/or Day 0, Week 2, Week 4, Week 6, Week 12, Week 24 and Week 48. Determination of analysis of collected samples for immunological endpoints will be determined on an ongoing basis throughout the study.

Peripheral Blood Immunogenicity Assessments: Whole blood will be obtained at Screening and/or Day 0 (34 mL at each visit or total 68 mL at pre-dose), and 34 mL at Week 2, Week 4 (pre-dose), Week 6, Week 12, Week 24 and Week 48 visits. Both Screening and/or Day 0 immunology samples (total 68 mL pre-dose samples) are required to enable all immunology testing. Serum samples (4 mL per visit from 10 mL of drawn whole blood) will be obtained at Screening and/or Day 0 (pre-dose), Week 2, Week 4 (pre-dose), Week 6, Week 12, Week 24 and Week 48 visit. Peripheral blood mononuclear cells (PBMCs) will be isolated.

The T and B cell immune responses to INO-4500 will be measured using assays that may include but are not limited to ELISA, ELISPOT, neutralization, assessment of immunological gene expression, assessment of immunological protein expression and flow cytometry.

Statistical Considerations:

Analysis populations will be:

The modified intention to treat (mITT) population includes all participants who receive at least one dose of the INO-4500 or SSC-0001. Participants in this sample will be analyzed by their original assigned dose regimen of INO-4500 or SSC-0001. The mITT population will be used to analyze secondary and exploratory immunological endpoints.

The safety analysis population includes all participants who receive at least one dose of INO-4500 or SSC-0001 administered by ID injection. Participants for this population will be grouped in accordance with the dose regimen of INO-4500 or SSC-0001 that were received. This population will be used for all safety analyses in the study.

Analysis of Primary Safety Endpoints. The primary analyses for this trial are safety analyses of treatment emergent adverse events (TEAE) and administration site reactions. TEAEs are defined for this trial as any AEs/SAEs that occur on or after Day 0. All TEAEs will be summarized by frequency, percentage and associated 95% Clopper-Pearson confidence interval. All TEAEs and related SAEs and related grade 3 AEs will be analyzed as the percentage difference and associated 95% confidence interval between those who received INO 4500 and their corresponding same regimen placebo. The frequencies will also be presented separately by dose number and will be depicted by system order class and preferred term. Additional frequencies will be presented with respect to maximum severity and relationship to IP. Multiple occurrences of the same AE in a single participant will be counted only once following a worst-case approach with respect to severity and relationship to IP. All serious TEAEs will also be summarized as above. AE duration will be calculated as AE stop date−AE start date+1 day. AEs and SAEs that are not TEAEs or serious TEAEs will be presented in listings. All of these primary safety analyses will be conducted on the participants in the safety population.

Analysis of Primary Immunology Endpoints. Levels of antigen specific binding antibody, LASV neutralization, and antigen specific cellular immune responses will be analyzed descriptively by dose regimen (study group) up to Week 6. Binding antibody levels will be analyzed by dose regimen using the geometric mean titers or medians and associated 95% confidence intervals up to Week 6. Week 6 percent neutralization and antigen specific cellular immune responses will be analyzed by dose regimen using medians, inter-quartile range and 95% confidence intervals. Percentage seroconversion (i.e., positive titer) and 95% Clopper-Pearson confidence intervals will be analyzed within each Study Group. The percentage difference between INO-4500 Study Groups will be calculated together with their exact 95% confidence intervals. Overall immune response within Study Group and relative immune response between study groups will be summarized using the same statistical methodology. All of these primary immunogenicity analyses will be conducted on the participants in the mITT population.

Exploratory Analyses. Levels of antigen specific binding antibody, LASV neutralization, and antigen specific cellular immune responses will be analyzed descriptively by dose regimen (study group) at Weeks 12, 24 and 48 in the same manner as described for the respective primary analyses.

Descriptive analysis of immunological gene expression, immunological protein expression, and additional analysis of all study weeks, may include mean/median and associated 95% confidence intervals, fold change, etc. where appropriate.

Exploratory ANCOVA and Logistic models may be fitted that include baseline, confounder variables such as age and gender to test the relationship between study group and different immune response variables.

Interim Analysis of Results:

Safety Results. INO-4500 (at a dose of 1 mg or 2 mg) is safe and well tolerated when administered ID and followed by EP. As of this analysis, a total of 191 participants (86.8%) experienced at least one (1) treatment-emergent Adverse Event (AE) during the clinical trial. Most TEAEs were Grade 1 or 2 in severity. There were five (5) participants (2.3%) with reported treatment-emergent serious adverse events (TESAEs), three (3) participants (1.4%) with reported Grade 3 TEAEs (2 cases of gastroenteritis [0.9%], 1 case of seizure [0.5%]) and one (1) participant with reported Grade 4 TEAE of headache (0.5%). All Grade 3 and 4 TEAEs and TESAEs were considered unrelated to the study treatment by the Investigator. There were no TEAEs that led to treatment discontinuation or study discontinuation.

The incidence of TEAEs is generally balanced across treatment groups. The most common TEAEs were injection site pain (reported in 140 participants overall [63.6%]) and injection site swelling (reported in 120 participants overall [54.5%]).

There were no reported cases of Sensoneural Hearing Loss (SNHL) (defined as hearing loss of 30 dB (or greater) in three sequential frequencies as measured with an audiogram for any participant during the study and no adverse event of hearing loss or difficulty for any study participant.

As of this analysis, a total of 104 participants (47.3%) experienced at least one (1) treatment-related AE as assessed by the Investigator. Dosing Regimen A (1 injection of 1 mg INO-4500+EP) had a total of 47 participants (53.4%) that experienced a total of 92 TEAEs assessed as related to the IP and/or EP. Dosing Regimen B (2 injections of 1 mg INO-4500+EP) had a total of 43 participants (48.9%) that experienced a total of 84 TEAEs assessed as related to IP and/or EP. The most common treatment-related TEAE for both Dosing Regimen A and B was injection site pain, reported in 53 participants (60.2%) and 58 participants (65.9%) respectively.

Dosing Regimen C (1 injection of SSC-0001+EP) had a total of 15 participants (68.2%) that experienced a total of 46 TEAEs assessed as related to IP and/or EP. Dosing Regimen D (2 injections of SSC-0001+EP) had a total of 17 participants (77.3%) that experienced a total of 60 TEAEs assessed as related to the IP and/or EP. The most common treatment-related TEAE for both Dosing Regimen C and D was injection site pain, reported in 13 participants (59.1%) and 14 participants (63.6%) respectively.

No clinically meaningful trends have been observed to date in clinical laboratory measurements or physical examination findings.

Immunogenicity Week 6 Results. Cellular responses were evaluated by IFNγ ELISpot assay following PBMC stimulation with two pools of LASV GPC peptides. IFNγ T cell responses were detected in 71% (37 out of 52) and 83% (40 out of 48) of subjects treated with 2 mg dose of INO-4500 (Group B) at WEEK 6 and WEEK 12, respectively (Table 5). In contrast, 16% (2 out of 12) and 15% (2 out of 13) of subjects in placebo control group D exhibited T cell responses at WEEK 6 and WEEK 12, respectively. At 1 mg dose of INO-4500 (Group A), cellular responses were detected in 64% (34 out of 53) and 50% (24 out of 48) of treated subjects at WEEK 6 and WEEK 12, respectively. Cellular reactivity was low in the corresponding placebo control group C (7% [1 out of 14] and 18% [2 out of 11] at WEEK 6 and WEEK 12, respectively). When the magnitude of cellular responses against the two pools of LASV GPC peptides in the different groups were compared, we found significantly higher reactivity at WEEK 6 and WEEK 12 in both INO-4500 treated groups (Groups A and B) compared to either baseline or respective placebo control groups (FIG. 5). For LASV pool 1, the mean IFNγ SFUs/million PBMCs was 57.5 and 38.9 at WEEK 6 and WEEK 12, respectively, for Group A, and 78.9 and 70.5 for Group B. For LASV pool 2, the mean IFNγ SFUs/million PBMCs was 89.3 and 92.7 at WEEK 6 and WEEK 12, respectively, for Group A, and 130.7 and 172.3 for Group B. These results showed higher cellular responses across the GPC region of the Lassa virus after vaccination with INO-4500 reaching more than 70% for the 2 mg dose group.

TABLE 5
LSV-002 GP ELISPOT Responders by Study Group
and Schedule Visit (mITT Population)

| | | INO-4500 + EP | | | |
|---|---|---|---|---|---|
| | | Group A | Group B | SSC-0001 + EP | |
| | | (1 mg) | (2 mg) | Group C | Group D |
| Visit | Statistic | (N = 88) | (N = 88) | (N = 22) | (N = 22) |
| WEEK 6 | % (n/N*) | 64.15 (34/53) | 71.15 (37/52) | 7.14 (1/14) | 16.67 (2/12) |
| | 95% CI | (49.80, 76.86) | (56.92, 82.87) | (0.18, 33.87) | (2.09, 48.41) |
| WEEK 12 | % (n/N*) | 50.00 (24/48) | 83.33 (40/48) | 18.18 (2/11) | 15.38 (2/13) |
| | 95% CI | (35.23, 64.77) | (69.78, 92.52) | (2.28, 51.78) | (1.92, 45.45) |
| Either | % (n/N*) | 68.97 (40/58) | 83.87 (52/62) | 18.75 (3/16) | 21.43 (3/14) |
| WEEK 6 | 95% CI | (55.46, 80.46) | (72.33, 91.98) | (4.05, 45.65) | (4.66, 50.80) |
| or 12 | | | | | |

*Percent responders based on n/N; n = number of responders at specific visit(s); N = number evaluable at specific visit(s). Clopper-Pearson method was used to calculate the 95% CI.

To examine the cellular immune response after INO-4500 vaccination and to support the IFNγ ELISpot data, a multiplex-based cytokine/chemokine assay (CRA) was used to quantify 14 different cytokines/chemokines in the supernatants harvested 20 h post-stimulation of PBMC with LASV pool of peptides. The pro-inflammatory cytokines IL-2, IFNγ, and TNFα, and the chemokines IFNγ-induced protein 10 (IP-10), and monokine-induced-by-IFN-γ(MIG) showed strong positive correlation with IFNγ ELISpot responses. In the 2 mg dose (Group B), 81% (43 out of 53) and 83% (41 out of 49) of subjects had a significant increases in these molecules at WEEK 6 and WEEK 12, respectively. At 1 mg dose (Group A), responses were detected in 65% (36 out of 55) and 66% (32 out of 48) of subjects at WEEK 6 and WEEK 12, respectively (Table 6). These data support the results of IFNγ ELISpot and the selection of 2 mg dose of INO-4500 to induce higher immune response.

TABLE 6
GP CRA Responders by Study Group and Scheduled Visit (mITT Population)

| | | INO-4500 + EP | | | |
|---|---|---|---|---|---|
| | | Group A | Group B | SSC-0001 + EP | |
| | | (1 mg) | (2 mg) | Group C | Group D |
| Visit | Statistic | (N = 88) | (N = 88) | (N = 22) | (N = 22) |
| WEEK 6 | % (n/N*) | 65.45 (36/55) | 81.13 (43/53) | 14.29 (2/14) | 33.33 (4/12) |
| | 95% CI | (51.42, 77,76) | (68.03, 90.56) | (1.78, 42.81) | (9.93, 65.11) |
| WEEK 12 | % (n/N*) | 66.67 (32/48) | 83.67 (41/49) | 16.67 (2/12) | 0.0 (0/13) |
| | 95% CI | (51.59, 79.60) | (70.34, 92.68) | (2.09, 48.41) | (0.00, 24.71) |

*Percent responders based on n/N; n = number of responders at specific visit(s); N = number evaluable at specific visit(s). Clopper-Pearson method was used to calculate the 95% CI

Antigen-specific IgG binding antibodies were measured by ELISA at Day 0, WEEK 6 and WEEK 12. Low levels of IgG binding antibodies were detected in all groups, however, subjects with a 2-fold increase above baseline at WEEK 6 and/or WEEK 12 were found in the INO-4500 vaccinated groups (Table 7). When only the subjects with positive quantification in the different groups were compared, it was found that the fold increase in Group B (2 mg dose) was significantly higher compared to Group A (1 mg dose) and that fold increase in both Group A and B were significantly higher than respective placebo groups.

TABLE 7
Antibody Responders: Two-fold Rise in Binding
Antibodies by Study Group and Scheduled Visit

| | | INO-4500 + EP | | | |
|---|---|---|---|---|---|
| | | Group A | Group B | SSC-0001 + EP | |
| | | (1 mg) | (2 mg) | Group C | Group D |
| Visit | Statistic | (N = 88) | (N = 88) | (N = 22) | (N = 22) |
| WEEK 6 | % (n/N*) | 1.18 (1) | 5.68 (5) | 0 | 0 |
| | 95% CI | (0.03, 6.38) | (1.87, 12.76) | (NA, NA) | (NA, NA) |
| WEEK 12 | % (n/N*) | 3.57 (3) | 9.30 (8) | 0 | 4.76 (1) |
| | 95% CI | (0.74, 10.08) | (4.10, 17.51) | (NA, NA) | (0.12, 23.82) |

*Percent responders based on n/N; n = number of responders at specific visit(s); N = number evaluable at specific visit(s). Clopper-Pearson method was used to calculate the 95% CI.

What is claimed:

1. A method of inducing a protective immune response against Lassa virus in a subject in need thereof, said method comprising administering a prophylactically effective amount of a nucleic acid molecule encoding the amino acid sequence of SEQ ID NO: 1 to the subject.

2. The method of claim 1, wherein the nucleic acid molecule comprises SEQ ID NO: 2.

3. The method of claim 1, wherein the nucleic acid molecule is an expression vector.

4. The method of claim 3, wherein the expression vector is a DNA plasmid.

5. The method of claim 4, wherein the DNA plasmid is pGX9808.

6. The method of claim 3, wherein the expression vector comprises a promoter, optionally a human cytomegalovirus promoter.

7. The method of claim 1, wherein the nucleic acid molecule is administered as a vaccine comprising a buffer.

8. The method of claim 7, wherein the buffer comprises sodium chloride and sodium citrate, optionally wherein the buffer comprises 150 mM sodium chloride and 15 mM sodium citrate at pH7.

9. The method of claim 1, wherein said administering comprises intradermal injection.

10. The method of claim 9, wherein said administering further comprises electroporation.

11. The method of claim 3, comprising administering a dose of 2 mg of the expression vector.

12. The method of claim 3, comprising administering an initial dose of 2 mg of the expression vector and a second dose of 2 mg of the expression vector.

13. The method of claim 12, comprising administering the second dose 23 to 33 days after the initial dose.

14. The method of claim 13, wherein the method provides a statistically significant increase in overall cellular response rate in the subject relative to baseline as measured by a Lassa virus glycoprotein precursor (LASV GPC) interferon-γ (IFN-γ) ELISpot assay six weeks and/or twelve weeks after the initial dose.

15. The method of claim 13, wherein the method provides a statistically significant increase in overall cellular response rate in a population of subjects administered the nucleic acid molecule as measured by a Lassa virus glycoprotein precursor (LASV GPC) interferon-γ (IFN-γ) ELISpot assay relative to a population of subjects not administered the nucleic acid molecule.

16. The method of claim 15, wherein the increase is about 40% to about 70%.

17. The method of claim 13, wherein the method induces:
a cellular immune response in about 70% of a population of subjects as measured by a Lassa virus glycoprotein precursor (LASV GPC) interferon-γ (IFN-γ) ELISpot assay six weeks following administration of the initial dose;
a cellular immune response in about 83% of a population of subjects as measured by a Lassa virus glycoprotein precursor (LASV GPC) interferon-γ (IFN-γ) ELISpot assay twelve weeks following administration of the initial dose;
a cellular immune response in about 80% of a population of subjects as measured by a multiplex-based cytokine/chemokine assay for interleukin-2 (IL2), interferon-γ (IFN-γ), tumor necrosis factor-α (TNF-α), IFN-γ-induced protein-10 (IP10), and monokine-induced-by-IFN-γ (MIG) six weeks following administration of the initial dose; and/or
a cellular immune response in about 80% of a population of subjects as measured by a multiplex-based cytokine/chemokine assay for interleukin-2 (IL2), interferon-γ (IFN-γ), tumor necrosis factor-α (TNF-α), IFN-γ-induced protein-10 (IP10), and monokine-induced-by-IFN-γ (MIG) twelve weeks following administration of the initial dose.

18. The method of claim 13, wherein the method provides no sensoneuronal hearing loss in the subject.
